(19) United States
(12) Patent Application Publication
(10) Pub. No.: US 2026/0207259 A1
(43) Pub. Date: Jul. 23, 2026

(54) NAVIGATED SYSTEM AND METHOD FOR IMPLANTING A SURGICAL IMPLANT

(71) Applicant: Kyniska Robotics SAS, Saint Martin d’Hères (FR)

(72) Inventors: Marie-Anne Chanrion, Saint Martin d’Hères (FR); Stéphane Lavallée, Saint Martin d’Hères (FR); Daniel Elizondo Moreno, Grenoble (FR)

(73) Assignee: Kyniska Robotics SAS, Saint Martin d’Hères (FR)

(21) Appl. No.: 19/447,804

(22) Filed: Jan. 13, 2026

Related U.S. Application Data
(60) Provisional application No. 63/744,581, filed on Jan. 13, 2025.

(51) Int. Cl. A61B 34/10; A61B 5/00; A61B 17/16; A61B 34/00; A61B 34/30

(52) U.S. Cl. CPC A61B 34/10; A61B 5/4528; A61B 17/1613; A61B 34/25; A61B 34/30; A61B 2034/104; A61B 2034/105; A61B 2034/108; A61B 2034/258

(57) ABSTRACT

A method includes acquiring a joint measurement data set for a joint, where the joint is defined by a first bone and a second bone. The joint measurement data set includes at least one of data on kinematics of the joint or data on laxities of the joint. The method includes, based on the joint measurement data set, determining at least one target implant parameter of an implant configured to be coupled to the first bone and the second bone. The implant is configured to, when implanted according to the target implant parameter, achieve at least one of a desired alignment, a desired spacing, or a desired loading of the joint.

### RELATED APPLICATION(S)

This application claims the benefit of and priority to U.S. Provisional Application No. 63/744,581, filed Jan. 13, 2025, the contents of which is incorporated herein by reference

### FIELD

This disclosure relates to systems and methods for guiding use of surgical implants having a linking member.

### BACKGROUND

Individuals can suffer from misalignment between two bones that define a joint. Such misalignment between the two bones can be painful, reduce quality of life, and result in further biomechanical issues and injuries in neighboring anatomy to the misalignment, such as cartilage in the knee joint. Osteotomy is a surgical procedure used to correct a misalignment of a joint, in particular in a lower limb (leg). To that end, an osteotomy procedure comprises cutting at least a portion of a bone defining the joint to create a bone hinge and moving the bone ends around the hinge to achieve a better alignment of the joint. Once the desired alignment has been achieved, an implant is placed between the bone ends to maintain them in the desired position. Osteotomy of a lower limb can be complex and require not only a precise diagnosis of the misalignment but also an accurate control of the surgical procedure itself. Moreover, osteotomy can be a highly invasive procedure.

In other circumstances, osteoarthritis can be observed in the knee joint, which could be caused by misalignment or other issues. This problem can be addressed with the pose of a prosthetic implant using total knee arthroplasty (TKA) or unicompartmental knee replacement (UKA), which are invasive methods with a relatively short lifespan of 15 to 20 years, and therefore are not recommended for young people.

An alternative to such invasive methods is the use of devices positioned just outside the affected joint capsule that can aid in absorbing part of the mechanical energy that normally passes through the cartilaginous tissue of the joint. In this way, the device could alleviate the overload in a particular zone of the joint and therefore reduce the pain experienced by the patient. Such devices need a proper positioning, which strongly depends on joint kinematics, in order to reach proper joint gap and force absorption. Manual methods for the positioning of such devices are prone to errors. Therefore, improved surgical systems and methods for positioning such implant devices are still needed.

### SUMMARY

The present disclosure relates to a method, including: acquiring a joint measurement data set for a joint, wherein: the joint is defined by a first bone and a second bone; and the joint measurement data set includes at least one of: data on kinematics of the joint; or data on laxities of the joint; and based on the joint measurement data set, determining at least one target implant parameter of an implant configured to be coupled to the first bone and the second bone, wherein: the implant includes: a first support configured to be fixed to the first bone; a second support configured to be fixed to the second bone; and a linking member extending between the first support and the second support; and the implant is configured to, when implanted on the joint according to the at least one target implant parameter, achieve at least one of a desired alignment, a desired spacing, or a desired loading of the joint.

In some embodiments, the present disclosure relates to a method, wherein the linking member is configured to absorb energy.

In some embodiments, the present disclosure relates to a method, wherein: the at least one target implant parameter includes an operating parameter of the linking member; and the operating parameter includes a characteristic affecting energy absorption of the linking member.

In some embodiments, the present disclosure relates to a method, wherein the at least one target implant parameter includes at least one of a placement of the first support on the first bone or a placement of the second support on the second bone.

In some embodiments, the present disclosure relates to a method, wherein: the placement of the first support on the first bone includes a pose of the first support on the first bone; and the placement of the second support on the second bone includes pose of the second support on the second bone.

In some embodiments, the present disclosure relates to a method, wherein: the placement of the first support on the first bone includes a pose of one or more anchor points of the first support on the first bone; and the placement of the second support on the second bone includes a pose of one or more anchor points of the second support on the second bone.

In some embodiments, the present disclosure relates to a method, wherein determining the pose of the one or more anchor points of the first support on the first bone or the pose of the one or more anchor points of the second support on the second bone includes constraining the pose of the one or more anchor points of the first support or the one or more anchor points of the second support to avoid limiting zones defined by one or more soft tissues or bones surrounding the joint.

In some embodiments, the present disclosure relates to a method, wherein the joint measurement data set includes the data on kinematics of the joint, and wherein acquiring the data on kinematics of the joint includes: manipulating at least one of the first bone or the second bone in a natural range of motion; and tracking a pose of the first bone or the second bone while manipulating the first bone or the second bone.

In some embodiments, the present disclosure relates to a method, wherein the joint measurement data set includes the data on kinematics of the joint, and wherein acquiring the data on kinematics of the joint includes: acquiring at least partial models of the first bone or the second bone; and applying a biomechanical model to the at least partial models of the first bone or the second bone, wherein the biomechanical model is adapted to patient-specific geometry of the first bone or the second bone.

In some embodiments, the present disclosure relates to a method, wherein the joint measurement data set includes the data on laxities of the joint, wherein acquiring the data on laxities of the joint includes: manipulating at least one of the first bone or the second bone in a constrained range of motion; and tracking a pose of the first bone or the second bone while manipulating the first bone or the second bone.

In some embodiments, the present disclosure relates to a method, wherein the joint measurement data set includes the data on laxities of the joint, wherein acquiring the data on laxities of the joint includes: acquiring at least partial models of the first bone or the second bone; and applying a biomechanical model to the at least partial models of the first bone or the second bone, wherein the biomechanical model is adapted to patient-specific geometry of the first bone or the second bone.

In some embodiments, the present disclosure relates to a method, wherein determining the at least one target implant parameter includes: determining, from the joint measurement data set, a pair of anisometric points (PAP) including a distance that satisfy a PAP length criteria, wherein the PAP includes a first point on the first bone and a second point on the second bone, and wherein the distance of the PAP is a distance between the first point and the second point as the joint is manipulated in natural ranges of motion, wherein the at least one target implant parameter is determined based on the PAP including the distance that satisfies the PAP length criteria.

In some embodiments, the present disclosure relates to a method, wherein the joint measurement data set includes the data on laxities of the joint, and wherein the PAP length criteria is determined based on the data on laxities of the joint.

In some embodiments, the present disclosure relates to a method, further including obtaining at least one of a partial model of the first bone or a partial model of the second bone, wherein determining the at least one target implant parameter is further based on the at least one of a partial model of the first bone or a partial model of the second bone.

In some embodiments, the present disclosure relates to a method, further including obtaining a model of the implant, wherein determining the at least one target implant parameter is further based on the model of the implant.

In some embodiments, the present disclosure relates to a method, further including obtaining the desired alignment, the desired spacing, or the desired loading of the joint, wherein determining the at least one target implant parameter is further based on the desired spacing, or the desired loading of the joint.

In some embodiments, the present disclosure relates to a method, further including: acquiring real-time tracking data, the real-time tracking data including: a pose of the first bone or the second bone; and at least one of: a pose of a surgical instrument; or a pose of a passive tool guide; and instructing, one or more of: a robotic system to drill one or more holes in the first bone or the second bone in accordance with the at least one target implant parameter and based on the real-time tracking data; the robotic system, based on instructions from a user presented with the real-time tracking data, to change a pose of the surgical instrument or the passive tool guide relative to the first bone or the second bone to drill one or more holes in accordance with the at least one target implant parameter; the robotic system to move, based on the at least one target implant parameter and the real-time tracking data, the passive tool guide to place the passive tool guide in a pose to guide the surgical instrument to drill one or more holes in the first bone or the second bone in accordance with the at least one target implant parameter; or a handheld robotic system to align, based on the at least one target implant parameter and the real-time tracking data, the surgical instrument with the first bone or the second bone for drilling one or more holes in the first bone or the second bone in accordance with the at least one target implant parameter.

In some embodiments, the present disclosure relates to a method, further including: acquiring real-time tracking data, the tracking data including a pose of the first bone or the second bone, a pose of a surgical instrument, and a pose of the implant device relative to each other; presenting, on a user interface, a display including a model of the implant, a model of the surgical instrument, and a model of at least one of the first bone or the second bone, in relation to each other in accordance with the real-time tracking data and the at least one target implant parameter, wherein a user is enabled to adjust a pose of the surgical instrument relative to the first bone or the second bone based on the user interface.

The present disclosure relates to a system, including: at least one processor; and at least one storage medium having encoded thereon executable instructions that, when executed by the at least one processor, cause the at least one processor to carry out a method, the method including: acquiring a joint measurement data set for a joint, wherein: the joint is defined by a first bone and a second bone; and the joint measurement data set includes at least one of: data on kinematics of the joint; or data on laxities of the joint; and based on the joint measurement data set, determining at least one target implant parameter of an implant configured to be coupled to the first bone and the second bone, wherein: the implant includes: a first support configured to be fixed to the first bone; a second support configured to be fixed to the second bone; and a linking member extending between the first support and the second support; and the implant is configured to, when implanted on the joint according to the at least one target implant parameter, achieve at least one of a desired alignment, a desired spacing, or a desired loading of the joint.

In some embodiments, the present disclosure relates to a system, wherein the linking member is configured to absorb energy.

In some embodiments, the present disclosure relates to a system, wherein: the at least one target implant parameter includes an operating parameter of the linking member; and the operating parameter includes a characteristic affecting energy absorption of the linking member.

In some embodiments, the present disclosure relates to a system, wherein the at least one target implant parameter includes at least one of a placement of the first support on the first bone or a placement of the second support on the second bone.

In some embodiments, the present disclosure relates to a system, wherein: the placement of the first support on the first bone includes a pose of the first support on the first bone; and the placement of the second support on the second bone includes pose of the second support on the second bone.

In some embodiments, the present disclosure relates to a system, wherein: the placement of the first support on the first bone includes a pose of one or more anchor points of the first support on the first bone; and the placement of the second support on the second bone includes a pose of one or more anchor points of the second support on the second bone.

In some embodiments, the present disclosure relates to a system, wherein determining the pose of the one or more anchor points of the first support on the first bone or the pose of the one or more anchor points of the second support on the second bone includes constraining the pose of the one or more anchor points of the first support or the one or more anchor points of the second support to avoid limiting zones defined by one or more soft tissues or bones surrounding the joint.

In some embodiments, the present disclosure relates to a system, wherein the joint measurement data set includes the data on kinematics of the joint, and wherein acquiring the data on kinematics of the joint includes: manipulating at least one of the first bone or the second bone in a natural range of motion; and tracking a pose of the first bone or the second bone while manipulating the first bone or the second bone.

In some embodiments, the present disclosure relates to a system, wherein the joint measurement data set includes the data on kinematics of the joint, and wherein acquiring the data on kinematics of the joint includes: acquiring at least partial models of the first bone or the second bone; and applying a biomechanical model to the at least partial models of the first bone or the second bone, wherein the biomechanical model is adapted to patient-specific geometry of the first bone or the second bone.

In some embodiments, the present disclosure relates to a system, wherein the joint measurement data set includes the data on laxities of the joint, wherein acquiring the data on laxities of the joint includes: manipulating at least one of the first bone or the second bone in a constrained range of motion; and tracking a pose of the first bone or the second bone while manipulating the first bone or the second bone.

In some embodiments, the present disclosure relates to a system, wherein the joint measurement data set includes the data on laxities of the joint, wherein acquiring the data on laxities of the joint includes: acquiring at least partial models of the first bone or the second bone; and applying a biomechanical model to the at least partial models of the first bone or the second bone, wherein the biomechanical model is adapted to patient-specific geometry of the first bone or the second bone.

In some embodiments, the present disclosure relates to a system, wherein determining the at least one target implant parameter includes: determining, from the joint measurement data set, a pair of anisometric points (PAP) including a distance that satisfy a PAP length criteria, wherein the PAP includes a first point on the first bone and a second point on the second bone, and wherein the distance of the PAP is a distance between the first point and the second point as the joint is manipulated in natural ranges of motion, wherein the at least one target implant parameter is determined based on the PAP including the distance that satisfies the PAP length criteria.

In some embodiments, the present disclosure relates to a system, wherein the joint measurement data set includes the data on laxities of the joint, and wherein the PAP length criteria is determined based on the data on laxities of the joint.

In some embodiments, the present disclosure relates to a system, wherein the method further includes obtaining at least one of a partial model of the first bone or a partial model of the second bone, wherein determining the at least one target implant parameter is further based on the at least one of a partial model of the first bone or a partial model of the second bone.

In some embodiments, the present disclosure relates to a system, wherein the method further includes obtaining a model of the implant, wherein determining the at least one target implant parameter is further based on the model of the implant.

In some embodiments, the present disclosure relates to a system, wherein the method further includes obtaining the desired alignment, the desired spacing, or the desired loading of the joint, wherein determining the at least one target implant parameter is further based on the desired spacing, or the desired loading of the joint.

In some embodiments, the present disclosure relates to a system, further including a robotic arm having a surgical instrument positioned thereon, wherein the method further includes instructing the robotic arm to move the surgical instrument relative to the first bone or the second bone in accordance with the at least one target implant parameter.

In some embodiments, the present disclosure relates to a system, further including a robotic arm having a surgical instrument positioned thereon, wherein the method further includes instructing the robotic arm to move the surgical instrument relative to the first bone or the second bone to drill one or more holes in the first bone or the second bone in accordance with the at least one target implant parameter.

In some embodiments, the present disclosure relates to a system, further including a robotic arm having a passive tool guide positioned thereon, wherein the method further includes instructing the robotic arm to move the passive tool guide to place the passive tool guide in a pose to guide a surgical instrument to drill one or more holes in the first bone or the second bone in accordance with the at least one target implant parameter.

In some embodiments, the present disclosure relates to a system, further including a handheld robotic surgical tool, wherein the method further includes aligning, based on the at least one target implant parameter, the handheld surgical tool with the first bone or the second bone for drilling one or more holes in the first bone or the second bone in accordance with the at least one target implant parameter.

The present disclosure relates to at least one computer-readable storage medium having encoded thereon executable instructions that, when executed by at least one control circuit, cause the at least one control circuit to carry out a method, the method including: acquiring a joint measurement data set for a joint, wherein: the joint is defined by a first bone and a second bone; and the joint measurement data set includes at least one of: data on kinematics of the joint; or data on laxities of the joint; and based on the joint measurement data set, determining at least one target implant parameter of an implant configured to be coupled to the first bone and the second bone, wherein: the implant includes: a first support configured to be fixed to the first bone; a second support configured to be fixed to the second bone; and a linking member extending between the first support and the second support; and the implant is configured to, when implanted on the joint according to the at least one target implant parameter, achieve one or more of a desired alignment, a desired spacing, or a desired loading of the joint.

In some embodiments, the present disclosure relates to an at least one computer-readable storage medium, wherein the linking member is configured to absorb energy.

In some embodiments, the present disclosure relates to an at least one computer-readable storage medium, wherein: the at least one target implant parameter includes an operating parameter of the linking member; and the operating parameter includes a characteristic affecting energy absorption of the linking member.

In some embodiments, the present disclosure relates to an at least one computer-readable storage medium, wherein the at least one target implant parameter includes at least one of a placement of the first support on the first bone or a placement of the second support on the second bone.

In some embodiments, the present disclosure relates to an at least one computer-readable storage medium, wherein: the placement of the first support on the first bone includes a pose of the first support on the first bone; and the placement of the second support on the second bone includes pose of the second support on the second bone.

In some embodiments, the present disclosure relates to an at least one computer-readable storage medium, wherein: the placement of the first support on the first bone includes a pose of one or more anchor points of the first support on the first bone; and the placement of the second support on the second bone includes a pose of one or more anchor points of the second support on the second bone.

In some embodiments, the present disclosure relates to an at least one computer-readable storage medium, wherein determining the pose of the one or more anchor points of the first support on the first bone or the pose of the one or more anchor points of the second support on the second bone includes constraining the pose of the one or more anchor points of the first support or the one or more anchor points of the second support to avoid limiting zones defined by one or more soft tissues or bones surrounding the joint.

In some embodiments, the present disclosure relates to an at least one computer-readable storage medium, wherein the joint measurement data set includes the data on kinematics of the joint, and wherein acquiring the data on kinematics of the joint includes: manipulating at least one of the first bone or the second bone in a natural range of motion; and tracking a pose of the first bone or the second bone while manipulating the first bone or the second bone.

In some embodiments, the present disclosure relates to an at least one computer-readable storage medium, wherein the joint measurement data set includes the data on kinematics of the joint, and wherein acquiring the data on kinematics of the joint includes: acquiring at least partial models of the first bone or the second bone; and applying a biomechanical model to the at least partial models of the first bone or the second bone, wherein the biomechanical model is adapted to patient-specific geometry of the first bone or the second bone.

In some embodiments, the present disclosure relates to an at least one computer-readable storage medium, wherein the joint measurement data set includes the data on laxities of the joint, wherein acquiring the data on laxities of the joint includes: manipulating at least one of the first bone or the second bone in a constrained range of motion; and tracking a pose of the first bone or the second bone while manipulating the first bone or the second bone.

In some embodiments, the present disclosure relates to an at least one computer-readable storage medium, wherein the joint measurement data set includes the data on laxities of the joint, wherein acquiring the data on laxities of the joint includes: acquiring at least partial models of the first bone or the second bone; and applying a biomechanical model to the at least partial models of the first bone or the second bone, wherein the biomechanical model is adapted to patient-specific geometry of the first bone or the second bone.

In some embodiments, the present disclosure relates to an at least one computer-readable storage medium, wherein determining the at least one target implant parameter includes: determining, from the joint measurement data set, a pair of anisometric points (PAP) including a distance that satisfy a PAP length criteria, wherein the PAP includes a first point on the first bone and a second point on the second bone, and wherein the distance of the PAP is a distance between the first point and the second point as the joint is manipulated in natural ranges of motion, wherein the at least one target implant parameter is determined based on the PAP including the distance that satisfies the PAP length criteria.

In some embodiments, the present disclosure relates to an at least one computer-readable storage medium, wherein the joint measurement data set includes the data on laxities of the joint, and wherein the PAP length criteria is determined based on the data on laxities of the joint.

In some embodiments, the present disclosure relates to an at least one computer-readable storage medium, wherein the method further includes obtaining at least one of a partial model of the first bone or a partial model of the second bone, wherein determining the at least one target implant parameter is further based on the at least one of a partial model of the first bone or a partial model of the second bone.

In some embodiments, the present disclosure relates to an at least one computer-readable storage medium, wherein the method further includes obtaining a model of the implant, wherein determining the at least one target implant parameter is further based on the model of the implant.

In some embodiments, the present disclosure relates to an at least one computer-readable storage medium, wherein the method further includes obtaining the desired alignment, the desired spacing, or the desired loading of the joint, wherein determining the at least one target implant parameter is further based on the desired spacing, or the desired loading of the joint.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A depicts an illustrative knee misalignment, according to one or more embodiments herein;

FIG. 1B depicts an illustrative knee joint unloading with an implant device, according to one or more embodiments herein;

FIG. 1C depicts an illustrative method for developing a surgical plan, according to one or more embodiments herein;

FIG. 2 depicts an illustrative method for developing a surgical plan, according to one or more embodiments herein;

FIG. 3A depicts an illustrative implant device, according to one or more embodiments herein;

FIG. 3B depicts an illustrative implant device fixed to a patient's knee joint, according to one or more embodiments herein;

FIG. 4A depicts an illustrative joint in a relaxed position, according to one or more embodiments herein;

FIG. 4B depicts an illustrative manipulation the joint depicted in FIG. 4A, according to one or more embodiments herein;

FIG. 5A-C depict an illustrative determination of characteristic points, according to one or more embodiments herein;

FIG. 6 depicts an illustrative knee joint with limiting zones of soft tissues of interest, according to one or more embodiments herein;

FIG. 7 depicts an illustrative surgical system and environment for planning a surgical procedure, according to one or more embodiments herein;

FIG. 8A depicts an illustrative surgical system for executing the surgical plan, according to one or more embodiments herein;

FIG. 8B depicts an illustrative surgical system for executing the surgical plan, according to one or more embodiments herein;

FIG. 8C depicts an illustrative surgical system for executing the surgical plan, according to one or more embodiments herein;

FIG. 9A depicts an illustrative robotic arm equipped with a surgical tool, according to one or more embodiments herein;

FIG. 9B depicts an illustrative robotic arm whose end effector is a passive tool guide, according to one or more embodiments herein;

FIG. 10 depicts an illustrative method for executing a surgical plan, according to one or more embodiments herein; and

FIG. 11 depicts a block diagram of an example computing system for determining a surgical plan for implanting an implant device and executing one or more surgical techniques.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure provides systems and methods for planning and executing a surgical procedure on a joint (to alleviate pain or reconstruct the joint, for instance). The surgical procedure discussed herein can generally be a procedure to adjust the alignment of two bones defining a joint, adjust a spacing between the two bones defining the joint, or adjusting the load (e.g., unloading) of the joint.

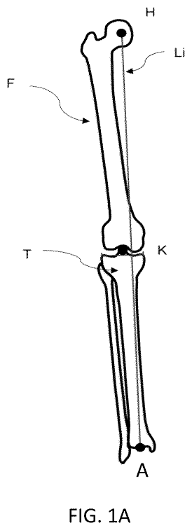

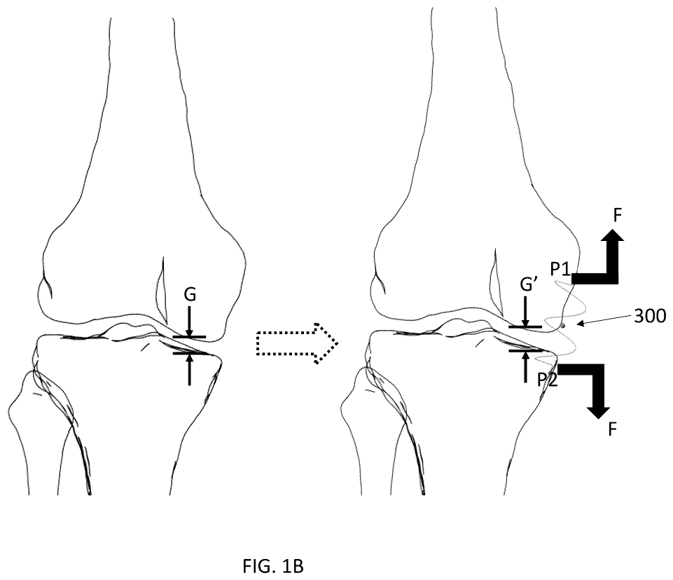

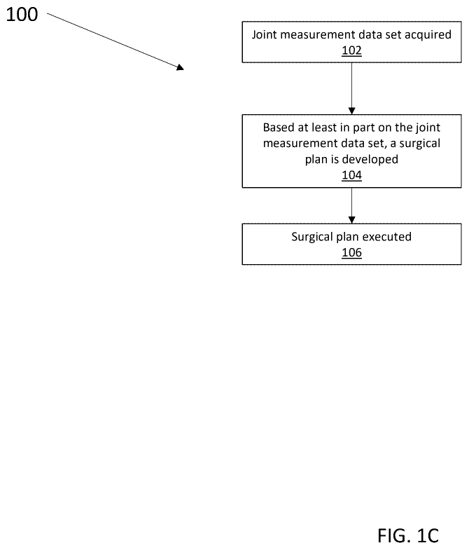

With respect to adjusting the alignment of two bones defining a joint, reference is made briefly to FIG. 1A, which shows an example of a knee misalignment. Specifically, FIG. 1A depicts a varus alignment of the knee (defined by the tibia T and femur F), where the initial weight-bearing line Li (also called Mikulicz line) that passes through the hip and ankle centers H, A, respectively, is located inwardly with respect to the knee center K. In some embodiments, the present disclosure provides systems and methods for re-aligning the knee, for instance, such that the weight-bearing line passes through the hip center H, the ankle center A, and the knee center K.

The systems and methods described herein for adjusting a joint (e.g., adjusting an alignment, spacing, or loading of the joint) can incorporate an implant device that includes a first support to be coupled to the first bone (e.g., the femur), a second support to be coupled to the second bone (e.g., tibia), and a linking member (e.g., a spring, a shock absorber, or an elastic material, which can be confined in a device or compartment, for instance) extending between the first and second supports. Such implant device is also called an energy-absorption device (EAD) 300 in the following discussion. In some embodiments, the linking member can be configured to absorb (e.g., store or dampen) energy to unload the joint. In some embodiments, an operating parameter (e.g., a tension, a stiffness, or an elasticity modulus) of the linking member can be selected to achieve a desired loading (e.g., unloading) of the joint, spacing of the joint, or alignment of the joint. A desired loading or unloading of the joint can be, for instance, a certain post-operative joint loading when the patient is in one or more respective positions (e.g., standing upright, standard gait, walking up stairs, etc.). In some embodiments, the present disclosure provides systems and methods for increasing, or otherwise adjusting, the space between the first bone and the second bone (to reduce bone on bone contact at the joint, for instance). In some embodiments, the linking member can be configured to bias the first and second bones. In some embodiments, an operating parameter of the linking member can be selected to achieve a desired spacing of the joint. The implant device can unload, re-align, or adjust the spacing of the joint.

Referring to FIG. 1B, an example of a joint unloading is shown. Specifically, FIG. 1B depicts a tibiofemoral joint with a specific joint gap G. By using an implant device 300, a force F is applied on the femur and tibia, unloading the joint with a new joint gap G′.

Still referring to FIG. 1B, systems and methods are proposed herein to plan the surgical positioning points P1 and P2 of the implant device 300. In some embodiments, the placement, or positioning (e.g., positioning points P1 and P2), of the first and second supports of the device on the first and second bones, respectively, and a setting of an operating parameter (e.g., a tension, stiffness, or elasticity) of the linking member can be selected to achieve a desired alignment of the joint, spacing of the joint, or loading of the joint.

Current methods in the field for adjusting the alignment, spacing, or loading the two bones defining a joint are manually intensive for surgeons and heavily rely on the surgeon's expertise to plan and execute the surgery. Specifically, currently in the field, a trial and error process is employed by the surgeon to couple an implant device 300 to a joint to desirably adjust the spacing, loading, or alignment of the joint. Such procedures are patient-specific due to their dependance of the patient's joint kinematics to achieve the desired joint unloading, alignment, or spacing goal. Specifically, current methods involve a surgeon coupling the implant device to first and second bones of a joint at a first set of points and assessing how the implant device affects the alignment, spacing, or loading of the joint when the implant is coupled to the joint at the first set of points. For instance, in some cases, the surgeon may manually assess how the implant device affects the alignment, spacing, or loading of the joint through manipulation of the joint (e.g., bones defining the joint) with the implant device coupled to the joint at the first set of points. If the desired alignment, spacing, or loading doesn't appear to be achieved, the surgeon then couples the implant device to the first and second bones of the joint at a second set of points and assesses how the implant device affects the alignment, spacing, or loading of the joint when the implant is coupled to the joint at the second set of points. This trial and error process can continue iteratively until the surgeon is satisfied with the alignment, spacing, or loading of the joint. This can increase the length of procedures, reduce the efficacy of the procedure, and increase patient trauma. Similarly, current methods can involve the surgeon performing a trial and error process with respect to the operating parameter (e.g., tensioning, stiffness, or elasticity) of the linking member of the implant device. Specifically, current methods involve a surgeon coupling the implant device to the joint with the linking member having a first operating parameter and assessing how the implant device affects the alignment, spacing, or loading of the joint when the linking member has the first operating parameter. For instance, in some cases, the surgeon may manually assess how the implant device affects the alignment, spacing, or loading of the joint through manipulation of the joint (e.g., bones defining the joint) with the linking member having the first operating parameter. If the desired alignment, spacing, or loading doesn't appear to be achieved, the surgeon then adjusts the operating parameter of the linking member and assesses how the implant device affects the alignment, spacing, or loading of the joint when the implant is coupled to the joint with the linking member having the second operating parameter. In some cases, the surgeon can iteratively adjust both the points the implant device is coupled to the joint and the operating parameter of the linking member at each step of the trial and error process until it is determined that a desired spacing, loading, or alignment is achieved at the joint.

The systems and methods described herein improve over current systems and methods, offering a computer-assisted surgical procedure that can be autonomously, or semi-autonomously, planned and executed. Specifically, the systems and methods described herein provide for the autonomous, or semi-autonomous, determination of the placement of an implant device on the two bones and the autonomous, or semi-autonomous, determination of a setting or operating parameter that the implant device should be configured to for adjusting the alignment, loading or spacing of a joint (e.g., re-aligning, unloading, or spacing the bones of the joint).

Referring now to FIG. 1C, a method 100 for planning and executing a surgical procedure is depicted. In some embodiments, in step 102 of the method 100, a joint measurement data set can be acquired either pre-operatively or intra-operatively. In some embodiments, the joint measurement data set can be acquired pre-operatively using computational methods based on the 3D patient-specific geometry of the joint and a biomechanical model that is adapted to the 3D patient-specific geometry and other data from the patient. In some embodiments, the joint measurement data set collected pre-operatively can include data on the kinematics or laxities of the joint. In some embodiments, the biomechanical model adapted to the 3D patient-specific geometry can reveal data on the kinematics or laxities of the joint. In some embodiments, the joint measurement data set can include the laxities of the joint. In some embodiments, to gather laxity data intraoperatively, the joint (e.g., the first bone and the second bone) can be manually held by the surgeon, using an arthrometer or a force-measuring device, or a robotic device in one or more constrained positions, where the joint is not free to relax. Using a tracking and localization system, data relating to the alignment or isometry of the joint in each constrained position can be gathered as the joint measurement data set. Such joint measurement data set can be indicative of the laxities of the patient's joint and the effects of the patient's soft tissues surrounding the joint on the joint alignment. In some embodiments, the joint measurement data set can include the kinematics of the joint (e.g., without measuring laxities). In some embodiments, to gather kinematic data intraoperatively, the kinematics can be gathered using a tracking and localization system along with passive movements of the joint initiated by the surgeon or with the aid of a robotic device. For example, a passive flexion of the knee, a passive flexion of the ankle, circular movements of the leg around the hip, or abductions of the leg could characterize the kinematics of such knee, ankle, or hip. In some embodiments, the joint measurement data set can include one or both of data acquired pre-operatively or data gathered intra-operatively. In some embodiments, the joint measurement data set can include one or both of data on joint laxities or data on joint kinematics.

In some embodiments, in step 104 of the method 100, based at least in part on the joint measurement data set, a surgical plan can be developed for adjusting the alignment, loading, or spacing of the joint. It should be appreciated that the surgical plan can be developed to achieve one or more of a re-aligning, unloading, or spacing of the joint. Moreover, it should be appreciated that in embodiments where the surgical plan is developed to unload or space the joint, the surgical plan can also be developed to achieve a target alignment of the joint. In some embodiments, the target alignment can be a native alignment of the joint that the surgeon wishes to maintain. The surgical plan can include a placement of the implant device on the first bone and the second bone, a placement of the fixation devices, such as screws, or an operating parameter of the implant device. The surgical plan can be tailored to a specific patient's joint measurement data set. For instance, the surgical plan can be tailored to a specific patient's biomechanics, joint laxity, soft tissues characteristics, or joint kinematics. Therefore, the surgical plan can, with greater efficacy, result in post-operatively achieving a desired alignment, loading, or spacing of the joint. In some embodiments, the surgical plan, including target implant parameters (as further discussed below) can be based on an initial joint geometry, for instance an initial alignment, spacing, or loading of the joint.

In some embodiments, in step 106 of the method 100, the surgical plan can be executed to adjust the alignment, spacing, or loading of the joint. For instance, the surgical plan can be executed to correct the alignment, spacing, or loading of the joint. Specifically, in a specific joint configuration (e.g. a knee flexion), the implant device can be implanted on the first bone and the second bone and the operating parameter can be selectively adjusted to adjust (e.g., correct) the alignment, spacing, or loading of the joint as desired in the rest of the joint configurations.

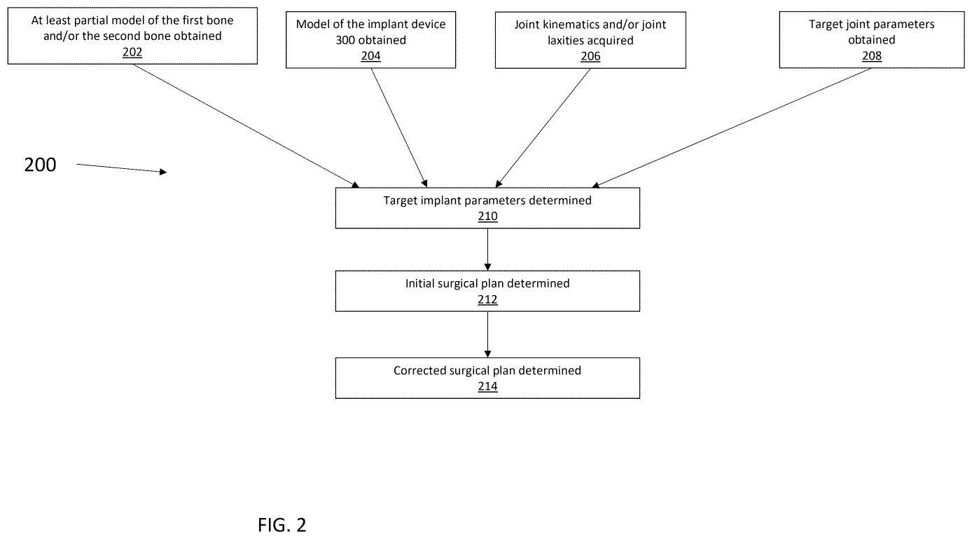

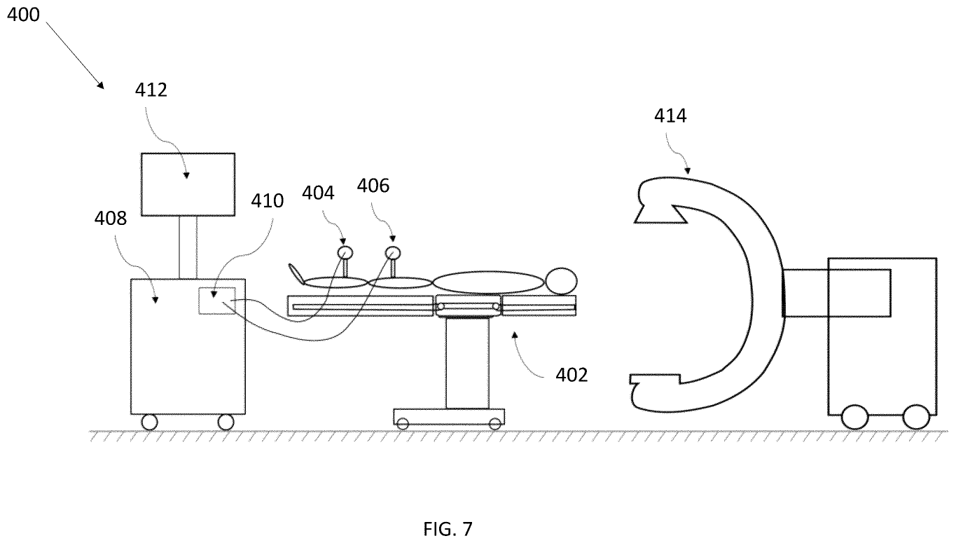

Steps 102 and 104 of the method 100 will be discussed in greater detail with respect to FIGS. 2-6. Step 106 of the method 100 will be discussed with greater detail with respect to FIGS. 7-10.

Referring to FIG. 2, a method 200 for planning a surgical procedure is depicted. In some embodiments, in step 202 at least a partial model of the first bone, the second bone, or both is obtained. In some embodiments, in step 204, a model of the implant device is obtained. In some embodiments, in step 206, the joint kinematics, joint laxities, or both are acquired. In some embodiments, in step 208, a target joint parameter is obtained. In some embodiments, in step 210, target implant parameters are determined. In some embodiments, in step 212, an initial surgical plan is determined. In some embodiments, in step 214, a corrected surgical plan is determined.

The method 200 can be performed either pre-operatively, intra-operatively or a hybrid combination between the two modes. In some embodiments, the surgical procedure can be a procedure to reconstruct a joint. For instance, the surgical procedure can adjust the alignment of (e.g., realign) a first and second bone with respect to one another, where the first and second bone define a joint, adjust a spacing between the first and second bone, or adjust a load of (e.g., unload) the joint. In some embodiments, adjusting the alignment of the first and second bone can increase or decrease the space or volume between the first bone and the second bone at the joint. Because the first and second bone define a joint, adjusting the alignment of the first and second bone, spacing of the first and second bone, or loading of the first and second bone, can include, and be described as, adjusting the alignment, spacing, or loading of the joint (defined by the first and second bone), respectively. It should be appreciated that the same holds for similar terms, such as alignment of the first and second bone and misalignment of the first and second bone, which can include, and be described as, alignment of the joint and misalignment of the joint, respectively. Other examples include implanting a device on the first bone or the second bone, which can include, and be described as, implanting a device on the joint (defined by the first bone and the second bone), and manipulating the first bone or the second bone, which can include, and be described as, manipulating the joint (defined by the first bone and the second bone). In some embodiments, the joint can be a knee joint, and the first and second bones can be the tibia and the femur. A surgical procedure performed on the knee joint for re-alignment, adjusting the spacing between, or unloading the tibia and femur will be discussed throughout the present disclosure in detail as an illustrative example. However, it should be appreciated that this is a non-limiting example and is discussed in detail only for illustrative purposes. For instance, it will be appreciated that the systems and methods of the present disclosure may also be used in other joints, including, but not limited to, shoulder (humerus and scapula), hip (femur and pelvis), elbow (humerus, radius, and ulna), spine (two or more vertebrae), wrist (ulna, radius, and carpals), ankle (tibia, fibula, and talus), and small joints in the hands and feet.

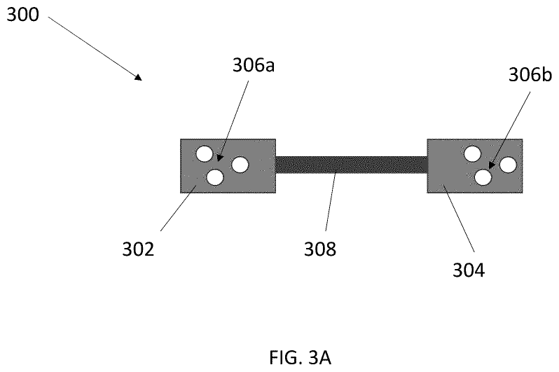

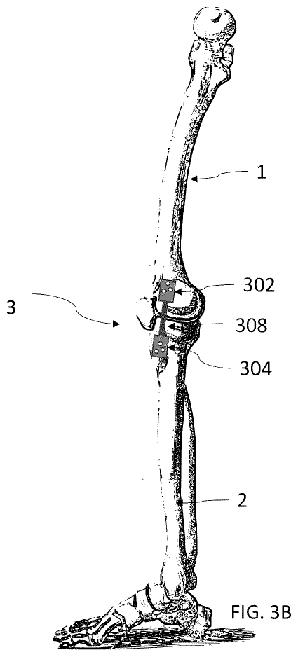

The method 200 for planning the surgical procedure can allow for a first determination on the desired placement (including, for instance, position and orientation) or operating parameter of the implant device configured to be coupled to the first bone and the second bone to adjust the alignment (e.g., realign), adjust the spacing between, or adjust the loading of (e.g., unload) the first bone and the second bone at the joint. The desired placement or operating parameter of the implant device can be the desired placement or operating parameter for post-operatively achieving a certain alignment, spacing, or loading/unloading (individually or collectively, generally referred to as a target joint parameter) of the joint. An example implant device 300 is depicted in FIG. 3A. In some embodiments, the implant device 300 includes a first support 302 configured to be coupled to the first bone and a second support 304 configured to be coupled to the second bone. In some embodiments, the desired placement of the implant device 300 includes a desired placement of the first support 302 on the first bone and a desired placement of the second support 304 on the second bone. The first support 302 and the second support 304 can each include one or more anchor points 306a and 306b, respectively, for coupling the first support 302 to the first bone and the second support 304 to the second bone. In some embodiments, the anchor points 306a, b can be throughbores, either threaded or unthreaded, sized to receive one or more fixation devices, such as screws, nails, pegs, Kirschner wires (K-wires), or any other fixation device configured to couple the first support 302 and the second support 304 to the first bone and the second bone, respectively. In some embodiments, the method 200 for planning the surgical procedure can allow for determining a desired placement (including, for instance, position, orientation, and depth of insertion into bone) of the fixation devices to be received through the anchor points 306a, b and into the first and second bones. The desired placement of the fixation devices can be the desired placement for post-operatively achieving an alignment, spacing, or loading/unloading (i.e., target joint parameter) of the joint.

Referring still to FIG. 3A, in some embodiments, the implant device 300 includes a linking member 308 extending between and coupled to the first support 302 and the second support 304. The linking member 308 can include one or more components configured to absorb (e.g., store or dampen) energy, such as a spring, hydraulic device, shock absorber, damper, flexible polymer, elastic material, and the like. In some embodiments, the linking member 308 can include one component, such as a spring, damper, etc. In some embodiments, the linking member 308 can include a plurality of components. In some embodiments, each component of the linking member 308 extends between and is coupled to the first support 302 and the second support 304. In some embodiments, a first component of the linking member 308 can be coupled to the first support 302, a second component of the linking member 308 can be coupled to the second support 304, and the first component and the second component of the linking member 308 can couple together. In some embodiments, the linking member 308 can control forces on the joint in question, such as by reducing certain loads on the joint. In some embodiments, the linking member 308 can bias the first bone and the second bone to achieve a desired alignment or spacing between the first bone and the second bone at the joint. Referring again to the method 200 shown in FIG. 2, in some embodiments, the method 200 includes determining one or more operating parameters, such as a stiffness, tension, elasticity, or other characteristic affecting energy absorption, of the linking member 308 of the implant device 300. Such operating parameters can be described as a target implant parameter. In some embodiments, the linking member 308 can be adjustable, such that a user can selectively adjust the one or more operating parameters of the linking member 308 to achieve a certain operative configuration of the linking member 308 and implant device 300. In some embodiments, the linking member 308 can be replaceable, or removably couplable to the implant device 300, such that a user can selectively couple a linking member 308 having desired operating parameters to the first support 302 and the second support 304.

Referring to FIG. 3B, the implant device 300 coupled to a first and second bone is depicted. Specifically, the first support 302 is coupled to a first bone 1, and the second support 304 is coupled to a second bone 2. In some embodiments, the linking member 308 can span the joint 3 defined by and between the first bone 1 and the second bone 2.

Referring again to FIG. 2, in a step 202 of the method 200, in some embodiments, an at least partial model of the first bone or the second bone can be obtained. In some embodiments, an at least partial 3D model of the first bone or the second bone can be obtained. In some embodiments, a full model of the first bone or the second bone can be obtained. In some embodiments, a full 3D model of the first bone or the second bone is obtained. In some embodiments, a full 3D model of the first bone or the second bone is not necessary to obtain. For instance, and with reference to a tibia and femur in relation to a knee joint to be realigned, merely as an example, a model of the whole tibia and femur may not be necessary, but only a model of the tibia and femur in the vicinity of the knee may be necessary to obtain. In some embodiments, only a model of the tibia and femur in the vicinity of the ankle, knee and/or hip (in particular to define the ankle, knee and hip centers) may be necessary to obtain.

Such at least partial models, which can be 3D models, of the first bone or the second bone can be obtained by various methods known by the skilled person. In some embodiments, such 3D bone models can be computed by automatic, semiautomatic, or manual segmentation of a 3D medical image of the patient's body or body part of interest (for instance the patient's leg or of portions of the patient's leg). In some embodiments, the bone models, which can be 3D models, can be obtained by palpation of the patient's bone using a tracked palpation probe. In some embodiments, the model of a bone, which can be a 3D model, can be computed based on a standard model of the bone, including characteristic points (e.g., landmarks and points of interest on the bone), by adjusting the standard bone model to at least one 2D or 3D medical image of the bone to match the patient's characteristic points, for example, with the aid of a statistical shape model (SSM) or with the aid of Atlas models that can be deformed in order to match the patient's model geometry. It should be appreciated that this is a non-limiting example of means to obtain the at least partial models, which can be 3D models, of the first bone or the second bone. Moreover, it should be appreciated that two or more of the above-described means can be combined to obtain the at least partial model, which can be 3D, of the first bone or the second bone. For example, in some embodiments, segmentation can be assisted by bone surface palpation.

The models of the first bone or the second bone, which can be 3D models, can allow for assessing multiple-plane deformities in three dimensions. In some embodiments, at step 202 of the method 200, the at least partial models of the first bone or the second bone, which can be 3D models, can be computed. In some embodiments, the at least partial models of the first bone or the second bone, which can be 3D models, may have been previously computed and stored in a database, for instance, and at step 202 of the method, the previously computed models can be obtained (e.g., from the database). In some embodiments, the at least partial model of the first bone or the second bone, which can be 3D, can include only an outer surface of the respective bone, and the bones can be considered as a rigid uniform volume.

In some embodiments, the at least partial model of the first bone or the second bone, which can be a 3D model, can also include a biomechanical model of the respective bones, which takes into account non-uniformities in the bone structure or the influence of soft tissues. For example, in some embodiments, a biomechanical model of the bone can include not only the outer surface of the bone but also several regions having different mechanical properties, such as cancellous bone regions and cortical bone regions, that do not deform the same way. In some embodiments, the model can include surrounding soft tissues such as cartilages, ligaments, tendons, muscles, veins, arteries and the like. In some embodiments, a biomechanical model can include the mechanical tension applied by ligaments or other soft tissues onto the bone, and/or conversely the mechanical tension applied by the bone onto soft tissues. Such additional biomechanical information can improve the efficacy of the surgical procedure.

In step 204 of the method 200, in some embodiments, a model of the implant device 300 is obtained. In some embodiments, the model of the implant device 300 can be a 3D model. The model of the implant device 300, which can be a 3D model, can include the outer surface of the implant device 300. In some embodiments, the model of the implant device 300, which can be a 3D model, can include the anchor points 306 a, b for coupling the first support 302 and the second support 304 to the first bone and second bone, respectively. In some embodiments, the model of the implant device 300, which can be a 3D model, is provided by the supplier or manufacturer of the implant device 300. In some embodiments, the model of the implant device 300, which can be a 3D model, can be available in a database of implant models.

In step 206 of the method 200, in some embodiments, a joint measurement data set can be acquired. In some embodiments, the joint measurement data set can include data on joint kinematics. The data on joint kinematics can include data on the motion of the joint (e.g., the first bone or the second bone) in the natural range of motion of the joint. The data on joint kinematics can include data on the motion of the joint (e.g., the first bone or the second bone) in the natural range of motion of the joint along one or more degrees of freedom (e.g. for a standard three-dimensional tracking system, six degrees of freedom can be acquired: three orthogonal translations and three orthogonal rotations), and where the motion of the joint implies the motion of either the first or second bone with respect to the fixed or mobile coordinate system of either the second or first bone. In some embodiments, the collected data on joint kinematics can be the transformation matrices (e.g. poses) of the bones using a common coordinate system (e.g., a camera, a emitter or a world coordinate system), and with an algebraic operation, the transformation of the motion of the first bone with respect to the second bone (e.g. changing the coordinate system of the first bone to be in the second bone) can be determined.

In some embodiments, the joint kinematics can be directly acquired intra-operatively using a tracking system of a computer-aided surgical system. For instance, the first bone or the second bone can have one or more tracking devices rigidly coupled thereto, and the tracking or localization system can be configured to track the position or orientation of the tracking devices (and therefore the bones coupled thereto). The user (e.g., a surgeon or another member of a medical staff) can then directly or indirectly manipulate the first bone (e.g. the body part, such as lower leg, of the first bone), or the second bone (e.g., the body part, such as lower leg, of the second bone) while keeping the other of the first or second bone (e.g. the body part of the first or second bone) in a known, fixed or mobile, position. In some embodiments, the user can manipulate the first bone or second around its full, natural range of motion. In some embodiments, the user can manipulate the first bone or the second bone in its natural range of motion or degrees of freedom (e.g., in its natural positions). The natural degrees of freedom or range of motion is defined by the degrees of freedom or range of motion that the first bone or second bone can be freely or willingly manipulated in or to (e.g., that the patient would be able to manipulate the first bone or the second bone in or to without the assistance of external forces). In some embodiments, the first or second bone can be manipulated with passive movements made using external assistance (e.g., a nurse or a physician), such as a passive flexion of the knee, a passive flexion of the ankle, circular movements of the leg around the hip, or hip abductions. The tracking system records the data during joint manipulation and provides the user with the motion of the first or second bone using the other of the first or second bone as a reference. In some embodiments, joint kinematics can further include data generated by direct manipulation of the relevant body parts of the user (e.g., the body part of the first bone or the body part of the second bone) with the aid of force and moment sensors. The kinematics data can then include the tracking kinematics data (e.g., position and orientation of first bone with respect to the second bone) coupled with force and moment data. In some embodiments, the bone manipulation can be automated using a robotic arm that will manipulate one of the bones (e.g. the first bone) and keep the other bone (e.g., second bone) in a known, fixed or mobile position. Examples of bone, or joint, manipulation to gather the kinematic data include the fixation of the thigh (femur bone, with a tracker attached) while creating a flexion-extension movement of the calf (tibia bone with a tracker attached). Another example is the tracking of the movement of two vertebrae in flexion-extension and lateral flexion in order to track the kinematics of this part of the spine.

The tracking or localization systems discussed herein are described as being configured to determine or track the position or orientation of a first object (e.g., the first bone) with respect to a second object (e.g., the second bone). In some embodiments, the term “pose” is used herein to include to refer to at least one of a position or an orientation of an object with respect to another object. It can, for instance, be represented as a transformation matrix between a reference system attached to a first object and a reference system attached to the other object. In some embodiments, as discussed herein, to locate or track an object can mean that it is possible to determine the pose of the concerned object. For instance, at least one of the position of the object (e.g., carrying one of a magnetic field transmitter or receiver) or the orientation of the object, with respect to another object (e.g., carrying another magnetic field transmitter or receiver) can be determined from data (e.g., magnetic data transmitted by the magnetic field transmitter and measured by a magnetic field receiver carried by the other object).

Still referring to step 206 of the method 200, in some embodiments the joint kinematics can be computed pre-operatively with the at least partial models of the first bone or second bone obtained in step 202 of the method 200. Using computer algorithms to calculate the movement of a bone with respect to another can be found in scientific literature. Examples of such include the calculation of the center of rotation of the femur with respect to the tibia during a flexion, and the calculation of the caudal-cranial displacement of the femur as a function of the radius of the femoral condyles. In some embodiments, the calculation of the joint kinematics can be computed pre-operatively with the at least partial models of the first bone or the second bone and a biomechanical model that is adapted to the patient-specific joint geometry. In some embodiments, the biomechanical model can be further adapted to other data acquired on the joint from a volume image of the joint (CT-scan, MRI). Such biomechanical model can include soft tissues (e.g. tendons, ligaments, muscles, cartilages, menisci, intervertebral disks, labrums, and the like) along with their mechanical properties. In some embodiments, muscle activation patterns can be simulated to compute the presumed movement of the joint and therefore compute the kinematics of the joint. The inclusion of the soft tissues comprises the gathering of the mechanical properties of such tissues, such as the elastic or viscoelastic properties, the fiber direction and the fiber pre-stretch data, among other properties that resemble the most the mechanical properties of the tissues present in the patient. Examples of using such biomechanical models include the use of an atlas biomechanical model whose bones geometries are deformed to target the geometries of the bones of the patient's joint, and then a computer simulation of the joint is executed to quantify the movements of the bones under anatomical loads. It should be appreciated that these examples are not exhaustive and other computational methods available in the state of the art can also be considered in this step in order to virtually quantify the joint kinematics.

Referring still to the step 206 of the method 200, in some embodiments, the joint measurement data set can include data on joint laxities. The data on joint laxities can include data on the motion of the joint (e.g., the first bone or the second bone) in a constrained range of motion of the joint. The data on joint laxities can include data on the motion of the joint (e.g., the first bone or the second bone) in the constrained range of motion of the joint along one or more degrees of freedom (e.g. for a standard three-dimensional tracking system, six degrees of freedom can be acquired: three orthogonal translations and three orthogonal rotations), and where the motion of the joint implies the motion of either the first or second bone with respect to the fixed or mobile coordinate system of either the second or first bone. In some embodiments, the collected data on joint laxities can be the transformation matrices (e.g. poses) of the bones using a common coordinate system (e.g., a camera, a emitter or a world coordinate system), and with an algebraic operation, the transformation of the motion of the first bone with respect to the second bone (e.g. changing the coordinate system of the first bone to be in the second bone) can be determined.

In some embodiments the joint laxities are physically acquired (e.g., intraoperatively). In some embodiments, the joint laxities can be computed pre-operatively. The joint laxities can be described as the movement of the joint in constrained degrees of freedom or ranges of motion (e.g., in constrained positions) as a function of a particular force or moment externally applied to the joint (e.g., to the first bone or second bone). The joint laxities can describe the state of the soft tissues such as ligaments, tendons, and the like in the joint. The constrained degrees of freedom or ranges of motion are defined by the degrees of freedom or ranges of motion that the first bone or second bone cannot be freely or willingly manipulated in or to (e.g., that the patient would be unable to manipulate the first or second bone in or to without the assistance of external forces). The constrained degrees of freedom or ranges of motion can include, for example, the varus-valgus rotation of the tibiofemoral joint, or the cranial-caudal displacement of the tibiotalar joint. An external force or moment can be needed to place the joint into a constrained position or within its constrained degrees of freedom, or range of motion. In some embodiments, the laxities can be obtained using an arthrometer pre-operatively or intra-operatively. In some embodiments, the laxities can be estimated using force and moment sensors attached to the tracked bones while the joint is manipulated (manually or with the aid of a device such as a robotic arm) in a constrained range of motion, or degree of freedom. In some embodiments, the laxities can be computed pre-operatively. In some embodiments the joint laxities can be computed pre-operatively with the at least partial models of the first bone or second bone obtained in step 202 of the method 200. In some embodiments, the calculation of the joint laxities can be computed pre-operatively with the at least partial models of the first bone or the second bone and a biomechanical model that is adapted to the patient-specific joint geometry. In some embodiments, biomechanical models can also include soft tissue data adapted either from segmentation of a Magnetic Resonance Image (MRI) of the patient, either from an atlas model that is adapted and deformed to match the geometry of the patient. Such soft tissues of the biomechanical model can include the tendons, ligaments, muscles, cartilages, menisci, invertebrate disks, labrums and the like. The inclusion of the soft tissues comprises the gathering of the mechanical properties of such tissues, such as the elastic or viscoelastic properties, the fiber direction and the fiber pre-stretch data, among other properties that resemble the most the mechanical properties of the tissues present in the patient. The biomechanical model can also include load data or boundary conditions such as it mimics the constrained movements related to the laxity. The biomechanical model can produce as an output the profile of the constrained movements and the necessary force to achieve such movements. It should be appreciated that any other method in the state of the art that allows the characterization of the laxities of the joint can be used in this step 206.

Examples of determining the laxities of the joint intraoperatively will now be discussed in greater detail. In some embodiments, with the patient on the operating table, the user (e.g., surgeon) manipulates the limb (e.g., the leg, joint, or first or second bone), to apply mechanical constraints to the joint (e.g., the knee) so as to bring the limb into one or a plurality of constrained positions (or move the limb within a constrained range of motion). As the joint is defined by the first and second bone, it should be appreciated the terms such as “applying mechanical constraints to the joint” and “a constrained position of the joint” can be used interchangeably with “applying mechanical constraints to the first bone or the second bone” and “constrained position of the first bone or the second bone,” respectively. For instance, to bring the limb into a constrained position (or within a constrained range of motion), the user can apply a force to at least one of the first bone or the second bone to move at least one of the first bone or the second bone about the joint to a constrained position (or within a constrained range of motion) that the patient, without the external force applied, would not be able to move the first bone or the second bone. The user can then hold the first bone or the second bone in the constrained position to prevent the first bone or the second bone from relaxing to a natural position. A plurality of constrained positions of the first bone or the second bone can be achieved depending on the direction and amplitude of forces applied to the first bone or the second bone by the user. The constrained positions (or constrained range of motion), and particularly the direction and amplitude of forces supplied by the user, can be selected by the user based on his clinical expertise. In some embodiments, the constrained positions (or constrained range of motion) can be selected in view of simulating the effect of laxities and soft tissues on the joint and can be defined by known clinical tests. Such clinical tests include, but are not limited to, the valgus stress test (or medial stress test), in which the user holds the patient's femur and pushes or pulls the tibia, and the Lachman test, in which the patient's knee is flexed to about 20°, the distal part of the femur is held by the surgeon with one hand, and an anterior traction of the tibia is exerted by the surgeon with the other hand.

In some embodiments, to collect the joint laxity or joint kinematics data, the position or orientation of the first bone or the second bone can be tracked with a tracking system. Specifically, trackers can be coupled to the first bone or the second bone and tracked with the tracking system. In some embodiments, characteristic points (e.g., landmarks and points of interest) on the first bone or the second bone are known in the coordinate system of at least one of the trackers fixed to the first bone and the second bone. Therefore, it is possible to track the position or orientation of the characteristic points over time when the user, for instance, manipulates the joint (e.g., the first bone or the second bone) into the one or more constrained or natural positions (or constrained or natural ranges of motion). In some embodiments, the positions or orientations of the first or second bones (including characteristic points on the bones) in each constrained or natural position (e.g., within constrained or natural ranges of motion) of the joint can be determined and recorded. In some embodiments, the positions or orientations of the first or second bones (including characteristic points on the bones) in each of the constrained or natural positions (e.g., within constrained or natural ranges of motion) can be recorded in a joint measurement data set. In some embodiments, the joint measurement data set includes data relating to constrained positions or ranges of motion of the joint in all possible directions in order to more fully gather data concerning the joint laxity and soft tissue of the patient. In some embodiments, the joint measurement data set includes data relating to natural positions or ranges of motions of the joint in all possible directions in order to more fully gather data concerning the joint kinematics of the patient.

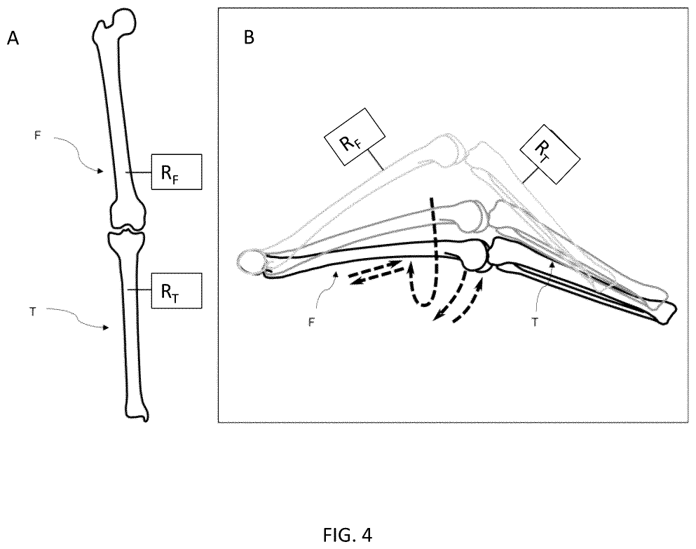

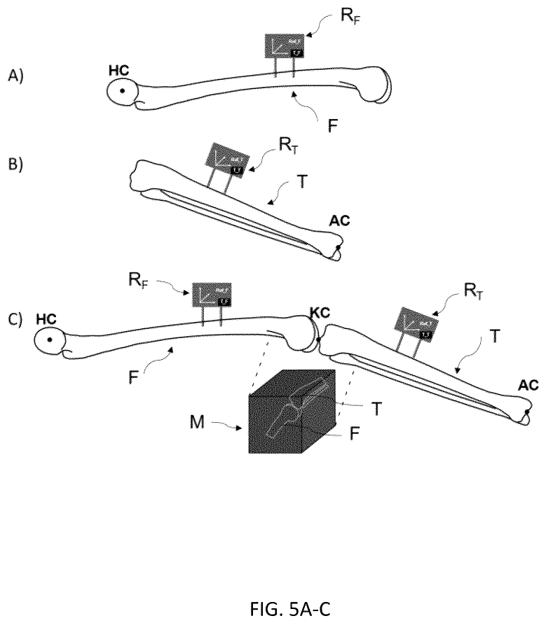

Referring to FIGS. 4A-B, first and second bones, particularly a femur F and tibia T, are depicted. FIG. 4A depicts the femur F and tibia T in a relaxed position, natural position, and marked with the femoral tracker RF and the tibial tracker RT fixed in the femur and tibia, respectively. It should be appreciated that “fixed,” as used herein in reference to the trackers on the first and second bones, means removably fixed. FIG. 4B depicts femur F and tibia T in a plurality of natural positions. In some embodiments, forces in the directions of the dashed arrows can be applied to the femur F or the tibia T to move the joint into a natural or constrained position. A localization system can track the femoral tracker RF and the tibial tracker RT to determine the positions of the first and second bones, such as femur F and tibia T (including characteristic points of the first and second bones) in each constrained or natural position (e.g., within the constrained or natural ranges of motion).

Referring again to examples of determining joint laxities of the joint, in some embodiments, each constrained position, or constrained range of motion, can be selected as being indicative or representative of the laxities of the joint and soft tissues surrounding the joint. In some embodiments, each constrained position, or constrained range of motion, can be selected by a control unit and presented to a user. In response, the user can apply mechanical constraints to the joint (e.g., the first bone or the second bone) to achieve the presented constrained positions, or constrained ranges of motion. In some embodiments, the constrained positions, or ranges of motion, to place the joint in can be determined as a function of natural positions or ranges of motion (for example, the constrained positions, or ranges of motion, can be determined from the joint kinematics data). In some embodiments, the joint laxities can be determined from the joint kinematics data. In some embodiments, the user can provide an input to the control unit, indicating the positions, or ranges of motion, at which the joint is constrained (e.g., the position of the first bone or the second bone). In some embodiments, at each constrained position, the position or orientation of the first and second bones (which can include characteristic points of the first and second bones) are determined and recorded by the control unit. In some embodiments, switching from one constrained position to the next one can be realized by the user pressing a button or a pedal, and in response, the control unit controlling a robotic arm or other hardware element can apply a mechanical constraint to the joint to achieve the next constrained position. In some embodiments, when the user has applied the mechanical constraint to the joint to achieve a desired constrained position, or range of motion, the surgeon can press a button or pedal to initiate a detection and recordation of the position or orientation of the first or second bones (which can include characteristic points of the first or second bones) at that constrained position, or within the constrained range of motion. In some embodiments, to gather laxity or kinematics data, the user or a hardware element controlled by the control unit can be given a determined amount of time to manipulate the first bone or the second bone (for example, 30 seconds) and during this time the positions or orientations of the first and second bones (which can include characteristic points on the first and second bones) can be determined and recorded by the control unit. At the end of the determined time, in some embodiments, the control unit can take into account the data acquired and check whether it constitutes a sufficient input to define or refine a model of the patient's joint. If not, the surgeon or control unit can manipulate the joint into one or more additional constrained or natural positions for further data collection.

Various methods for determining a set of characteristic points on the first bone or the second bone can be used. In some embodiments, the characteristic points can be acquired by palpation using a pointer carrying a tracker that is tracked by a localization system linked to the computer-aided surgical system relative to trackers carried (e.g., removably fixed) by the first bone and the second bone. In such a way, the position of each palpated point relative to at least one of the tibial tracker or femoral tracker can be determined. A coordinate system can be attached to each tracker, allowing the trackers to be tracked in real time by the localization system.

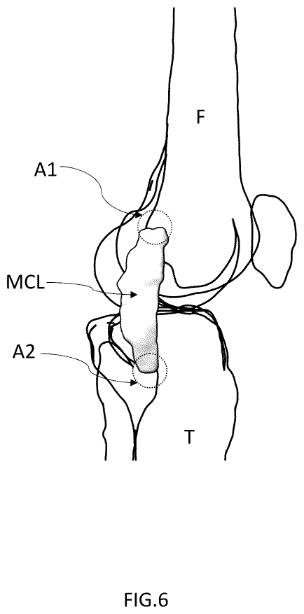

Referring now to FIGS. 5A-C, example characteristic points are shown according to exemplary embodiments, where the first bone and the second bone include a femur F and a tibia T. As shown in FIG. 5A a characteristic point located on the femur bone F, for instance the hip center HC, can be determined and located relative to a navigation tracker RF fixed on the femur bone F. It should be appreciated that “fixed,” as used herein in reference to the trackers on the first and second bones, means removably fixed. As shown in FIG. 5B, a characteristic point located on the tibia bone T, for instance the ankle center AC, can be determined and located relative to a navigation tracker RT fixed on the tibia bone T. As shown in FIG. 5C, around the knee joint, a partial 3D model M of the tibia T and the femur F can be obtained. This model can be obtained for instance from a 3D imaging acquisition, which for instance can be, but are not limited to: cone-beam computer tomography (CBCT), computer tomography (CT) scan or magnetic resonance imaging (MRI). In some embodiments, the 3D model M can be generated by any of the above-described methods of generation. Characteristic points on the 3D model M, for instance the knee center KC, can be determined and located relative to a navigation tracker fixed on the tibia bone T or on the femur bone F (e.g., the navigation tracker RF or the navigation tracker RT).

In some embodiments, the characteristic points of the first bone or the second bone can be determined on at least one 2D (bi-dimensional) (e.g. X-ray) or 3D (three-dimensional) (e.g. computer tomography or a magnetic resonance imaging) image whose position in the coordinate system of at least one tracker is known. In some embodiments, the 2D or 3D image is acquired in an intra-operative step. In some embodiments, the 2D or 3D image can be acquired pre-operatively. In such case, a registration is done between the pre-operative 2D or 3D image and the coordinate system of the tracker of at least one of the first bone and/or the second bone.

In some embodiments, the characteristic points can be identified based on a segmentation of the 2D or 3D image(s). For instance, the knee joint center can be located in at least one 2D or 3D image of the knee, via the analysis of one or more characteristic points found on the images. In particular, different characteristic points of the image can be used to define the knee center. In some embodiments, the knee center can be defined, for instance, as the center of the apex of the femoral notch, the midpoint of the femoral condyles, the center of tibial spines, the midpoint of soft tissue outline, or the midpoint of tibial plateaus. In some embodiments, the knee center can be the average of two or more points, such as the midpoint of the femoral condyles and the center of the soft tissue outline. In some embodiments, the characteristic points can be identified in the at least partial 3D model of the first bone or the second bone obtained in step 202 of the method 200. If several partial 3D models of the first bone or the second bone are obtained, they can be defined in a common coordinate system. In some embodiments, the characteristic points may be present on a model of the bone and the model can be adjusted to the patient's bone using known bone morphing techniques so as to register the characteristic points with a 2D or 3D image of the patient's bone.

In some embodiments, some characteristic points (for example, the center of a joint) can be determined by moving a limb, or a part of the limb, that includes the first bone or the second bone about the joint defined by the first bone and the second bone. For example, the knee joint center can be located by using a morpho-functional method using flexion-extension movements. In such a morpho-functional method, 3D positions and orientations of the femoral and tibial trackers can be recorded during the movement of the limb by the localization system, and an average rotation axis representing the flexion/extension can be estimated.

In some embodiments, any of the above-described methods can be combined to determine the characteristic points. For example, the hip, knee or ankle center can be initially obtained via the morpho-functional method and subsequently adjusted in view of information of the position of other characteristic points on gathered from 2D or 3D images. As another example, in some embodiments, a partial determination of the characteristic points based on an incomplete bone segmented medical image or an incomplete bone morphing can be supplemented by the palpation of other characteristic points with a tracked pointer. In some embodiments, the characteristic points can be labelled on the at least partial model of the first bone or the second bone. In some embodiments, the location of the characteristic points can be used to modify or improve the at least partial model of the first bone or the second bone.

In step 208 of the method 200, in some embodiments, a target joint parameter can be determined. In some embodiments, one or more computer systems can determine the target joint parameters based on some or all of the information gathered in steps 202-206 of the method 200. In some embodiments, a user (e.g., a surgeon or another member of a medical staff) can provide at least one input, including at least one target joint parameter. The target joint parameter can be one or more of the desired post-operative alignment between the first bone and the second bone at the joint defined by the first bone and the second bone, the desired post-operative spacing between the first bone and the second bone at the joint, or the desired post-operative loading of the joint. The at least one target joint parameter can include a desired post-operative isometry profile of the joint. The isometry profile may be considered a desired distance between characteristic points on the first bone and the second bone at a set of different flexion angles of the joint. In some embodiments, the target joint parameter(s) can be determined and input in a fully preoperative step. In some embodiments, the target joint parameter(s) can be determined and input intra-operatively (e.g., after an incision is made on the patient). One or more target joint parameters can be input by the user depending on the specific condition of the patient to be addressed. As non-limiting examples, the one or more target joint parameters can be a mechanical femorotibial angle (mFTA), a hip-knee-ankle angle (HKA) (e.g., angle between the mechanical axes of the femur and the tibia), a joint line convergence angle (JLCA) (e.g., angle made by a tangential line between the femoral condyles and the tibial plateau), a posterior tibial slope (PTS), a hip abduction angle (HAA), a tibial plafond inclination (TPI), a talar inclination angle (TIA), a lateral patellar tilt (LPT), or a lateral patellar shift (LPS).

In some embodiments, such target joint parameters are determined relative to a set of anatomical landmarks (also referred to as characteristic points) of the first bone or the second bone. In step 208 of the method 200, in some embodiments, a user (e.g. a surgeon or another member of a medical staff) can provide a desired gap in a joint side using the implant device 300 as a target joint parameter. Such gap can be estimated based on other joint parameters such as bones sizes or the joint measurement data set, including joint laxities or kinematics. In some embodiments, the target gap in the joint is provided by the implant device 300 manufacturer.

Still referring to step 208 of the method 200, the one or more target joint parameters can be determined by various techniques. In some embodiments, the target joint parameters can be determined based on 2D X-ray images of the first bone or the second bone, 3D images of the first bone or the second bone, or models of the first bone or the second bone. In some embodiments, the target joint parameters can be determined based on measurements of specific features, in the 2D or 3D images for instance, representative of the alignment, spacing, or loading of the first bone and the second bone. In some embodiments, the 2D or 3D images can be acquired pre-operatively. In such embodiments, the pre-operative images can be acquired with the patient generally in a standing position. In the step 208, a user can diagnose a misalignment of the patient's first bone and second bone, a misspacing between the first bone and the second bone at the joint, or a loading of the joint (which in some embodiments can be determined at least in part on the alignment and spacing of the joint). In the step 208, the user can consequently determine the one or more target joint parameters to be achieved by the surgical procedure to provide a desired alignment of the first bone and the second bone, a desired spacing between the first bone and the second bone at the joint, or a desired post-operative loading of the joint (e.g., a desired loading of the joint in one or more biomechanical positions (standing, walking, etc.)). In some embodiments, the 2D or 3D images can be acquired while the patient can generally be lying on the operating table.

Due to the length of the limb being studied, such as a leg, it may not be possible or suitable to obtain a single 2D or 3D image of the whole limb. Thus, several images of different parts of the limb can be acquired and recorded to allow determining target joint parameters of the first bone and the second bone.

It should be appreciated that the use of 2D or 3D images is not compulsory to determine the target joint parameters. For example, the localization and tracking system alone can be sufficient to determine the relative positions of the first bone and the second bone in all directions of a three-dimensional space, diagnose a misalignment, misspacing, or loading of the first bone and the second bone, and determine the one or more target joint parameters to be achieved by the surgical procedure to provide a desired alignment, spacing, or loading of the first bone and the second bone. Such approaches can reduce the patient's exposure to X-rays or other imaging modalities.

Still referring to step 208 of the method 200, in addition to the one or more target joint parameters, the input entered in step 208 can also include the type of surgical procedure to be performed. The type of surgical procedure includes the surgical approach which determines the tissues to be exposed during the surgery and therefore the surgical volume available to the surgical tools during the intervention. In some embodiments, the input provided by the user can be a validation of an automatic surgical plan including computer generated target joint parameters.

In a step 210 of the method 200, in some embodiments, target implant parameters can be determined. In some embodiments, the target implant parameters can be determined based on the joint measurement data set determined in step 206. In some embodiments, the target implant parameters can be determined based on one or more of the at least partial model of the first bone or the second bone determined in step 202, the model of the implant device 300 determined in step 204, the joint measurement data set determined in step 206, or the target joint parameters determined in step 208. In some embodiments, the target implant parameters can include the desired placement of the implant device 300 onto the first bone or the second bone. In some embodiments, the desired placement of the implant device 300 is determined based on one or more of the model (e.g., 3D model) of the implant device 300, the at least partial models (e.g., 3D models) of the first bone or the second bone, the characteristic points of the first bone or the second bone, the joint measurement data set (e.g., the joint kinematics or joint laxities), or the target joint parameters. The desired placement of the implant device 300 can be selected or determined to, at least in part, achieve the target joint parameters.

As mentioned above, the joint measurement data set can include recorded information on the tracked positions or orientations of the first and second bones in constrained positions, or ranges of motion, or in natural positions, or ranges of motions. Based on the localization, or tracking data, an alignment or spacing of the joint in each constrained or natural position, or ranges of motion, can be determined. In some embodiments, a native alignment or spacing of the joint in a relaxed (e.g., straight) position and an alignment or spacing of the joint determined in each constrained or natural position (or ranges of motion) the joint is manipulated into can be compared. In some embodiments, a distance between a first point on the first bone and a second point on the second bone can be determined at each constrained or natural position, or as the bones are moved in the constrained or natural ranges of motion. In some embodiments, the distance between the first point and the second point can be a Euclidean distance. In some embodiments, the distance between the first point and the second point can be a trace of the Euclidean distance between the first point and the second point. In some embodiments, the distance between the first point and the second point can be both a Euclidean distance and a trace of the Euclidean distance between the first point and the second point. In some embodiments, a distance between various pairs of points (with a first point being on the first bone, and a second point being on the second bone) can be determined at each constrained or natural position, or as the bones are moved in the constrained or natural ranges of motion. In some embodiments, based on the determined distances between pairs of points, a profile of the joint can be determined.

The localization of the points in the constrained or natural positions (or ranges of motion), the comparison between the alignment or spacing of the joint in the relaxed position and alignment or spacing of the joint in the one or more constrained or natural positions (or ranges of motion), the determined distances between the points in the constrained or natural positions (or ranges of motion) (e.g., joint profile) can indicate the effects of joint kinematics and joint laxity and surrounding soft tissue on the alignment, spacing, or loading of the joint. The determination on the effects of kinematics or laxity and soft tissues can prevent over-correction or under-correction of joint alignment, spacing, or loading during the surgical procedure. For example, for two different patients having a same joint alignment in the relaxed position of the joint and a same target joint parameter, the post-operative alignment of the joint may be different depending on the laxity of the patients' joints and the soft tissues surrounding the joints.

In some embodiments, the joint measurement data set can be applied to a function or algorithm that defines or modifies a model of the patient's joint (such as the 3D model or biomechanical model discussed above), and further determines the implant parameters based on the model of the patient's joint. As noted, the function or algorithm can determine the implant parameters based on the target joint parameter, or more specifically, to achieve the target joint parameter. In some embodiments, the model of the patient's joint can be used to determine the laxity of each ligament in the joint of interest. In some embodiments, the function or algorithm can be trained on data sets, including pre-operative joint profile (e.g., alignment/distance pattern), joint profile at constrained and natural positions (or ranges of motion), or post-operative joint profile, from various patients. In some embodiments, the function or algorithm can be continuously updated and refined based on machine learning methods. In some embodiments, after the surgical procedure is completed and before removing the trackers coupled to the first bone and the second bone, another measurement data set (e.g., localization of characteristic points, distances between pairs of points, joint profile, etc.) can be acquired with the surgeon manipulating the joint into natural or constrained positions (or ranges of motion). The post-operative measurement data set can then be used to train and refine the algorithm or function used to determine effects of laxities and soft tissues and the implant parameters. The post-operative measurement data set can be used to determine the efficacy of the surgical procedure, after completed, in achieving the target joint parameters.

In some embodiments, the target implant parameters can be determined based on the joint profile determined from the joint measurement data set. In some embodiments, the joint measurement data set can include the joint profile. That is, in some embodiments, the target implant parameter can be determined the based on the distances between one or more pairs of points (with a first point being on the first bone, and a second point being on the second bone) determined in constrained or natural positions, or as the bones are moved in the constrained or natural ranges of motion.

As mentioned above, in some embodiments, the target implant parameter can include a desired placement of the implant device 300 on the joint (e.g., on the first bone and second bone). In some embodiments, the desired placement of the implant device 300 can include a desired position of the first support 302 on the first bone and a desired position of the second support 304 on the second bone. In some embodiments, the desired placement of the implant device 300 can include a desired orientation of the first support 302 on the first bone and a desired orientation of the second support 304 on the second bone.

In some embodiments, the desired placement of the implant device 300 can be determined based, at least in part, on a protrusion of the implant device 300 from the surface of the first bone or the second bone. For instance, the desired placement of the implant device 300 can be determined to minimize a protrusion, which in some embodiments, can result in the outer surface of the first support 302 or the second support 304 being flush with an outer surface of the first bone or the second bone. The desired placement of the implant device 300 can include achieving any desired protrusion from the outer surfaces of the first bone or the second bone.

In some embodiments, the desired placement of the implant device 300 can be based, at least in part, on the position of the anchor points 306a,b. For instance, the desired placement of the implant device 300 can be determined such that each anchor point 306a,b of the implant device 300 is located over a surface of the first bone or the second bone. In some embodiments, the anchor points position can be constrained based on the other bones or soft tissues limiting zones, in a manner such that the anchor points avoid the limiting zones. The limiting zone, for instance, can be any area or volume of space occupied by at least a portion of another bone or soft tissue that the user does not want the implant device 300 to contact or otherwise interfere with. For example, the desired placement of the implant device 300 can be determined such that each anchor point 306a,b is not located in or over a limiting zone. Referring briefly to FIG. 6, an example of this is depicted where the implant device anchor points 306a,b are determined to avoid the limiting zone defined by the medial collateral ligament (MCL) insertion points A1 and A2. For an implant device 300 inducing a gap G by applying force F in the medial compartment of the knee, it could be placed parallel to the MCL and the anchor points 306a,b around the limiting zones A1 and A2. Such limiting zones can be acquired using 3D segmentation of 3D image (CT-scan if only bone, MRI if soft tissue), or using a tracked palpation probe intra-operatively linked to a computer-aided surgical system.

In some embodiments, the desired placement of the implant device 300 can be determined such that the first support 302 and the second support 304 of the implant device 300 are constrained based on limiting zones defined by other bones or soft tissue surrounding the joint. In some embodiments, the position of the first support 302 and the second support 304 can be constrained based on the other bones or soft tissues limiting zones, in a manner such that the first support 302 and the second support 304 avoid the limiting zones. The limiting zone, for instance, can be any area or volume of space occupied by at least a portion of another bone or soft tissue that the user does not want the implant device 300 to contact or otherwise interfere with.

In some embodiments, the desired placement of the implant device 300 can be based, at least in part, on predetermined instructions of use of the implant device 300. For instance, in some embodiments, the instructions can include a range of positions or orientations that the first support 302 should be placed on the first bone, a range of positions or orientations that the second support 304 should be placed on the second bone, and a range of distances that the first support 302 and the second support 304 should be from each other.

In some embodiments, the desired placement of the implant device 300 can be based, at least in part, on stress recovery. For instance, in some embodiments, the desired placement of the implant device 300 can be determined to maximize stress recovery. In some embodiments, the desired placement of the implant device 300 can be based, at least in part, on friction of the implant device 300 on surrounding soft tissue. For instance, in some embodiments, the desired placement of the implant device 300 can be determined to minimize friction on surrounding soft tissue. The determination of the friction on surrounding soft tissue can be based on a biomechanical model of the first bone and/or the second bone.

In some embodiments, the desired placement of the implant device 300 can be determined based on a contact surface area of the first support 302 or the second support 304 with the first or second bone, respectively. In some embodiments, the desired placement of the implant device 300 can be determined to maximize a contact surface area of the first support 302 or the second support 304 with the first or second bone, respectively.

One example embodiment of using the joint measurement data set to determine the target implant parameters follows. As mentioned above, the joint measurement data set can include a joint profile. The joint profile can map the distance between pairs of points (with one point on the first bone of the joint and the other point on the second bone of the joint) as a function of the natural or constrained positions, or ranges of motion (e.g., as the joint is moved in the natural or constrained ranges of motion). As mentioned above, the distance or length can be a Euclidean distance or a trace of the Euclidean distance. A pair of points, where the distance between the points varies as a function of the natural or constrained positions (or ranges of motion), are a pair of anisometric points (PAP). A PAP, satisfying one or more requirements, can define or indicate points on the first bone and the second bone where implant device 300 should be placed. In some embodiments, at a first step, an initial PAP length can be obtained from the manufacturer of the implant device 300 or set by the user (e.g., surgeon). The initial PAP length can inform the user to determine a PAP, where the distance between the points defining the PAP, during motion of the joint, varies at least by the initial PAP length. In some embodiments, the initial PAP length can be the expected range of motion of the implant device 300. For instance, if the implant device 300 were expected to have a range of motion of 4 mm in length and to be positioned on the medial side of the knee, a PAP that varies by at least 4 mm during a passive knee flexion of 0° (extension) to 90° should be determined to guide placement of the implant device 300. As another example, an implant device 300 expected to have a range of motion of 5 mm in length to be positioned between two vertebrae in their posterior aspect must have a PAP determined whose length is 5 mm during flexion, extension, side bending and rotation of the vertebral column.

However, the initial PAP length may not take into account data on joint kinematics or joint laxities. Therefore, in a second step, in some embodiments, the joint measurement data set, and specifically, data on the laxity of the joint, can be gathered as described above and used to determine a modified PAP length. That is, depending on the patient-specific joint laxities, the initial PAP length can be modified to account for the patient's joint having increased or decreased flexibility or range of motion than the average, as indicated by the laxity data gathered in the joint measurement data set. Therefore, merely as an example, the initial PAP length can be modified from 4 mm to 5 mm or 3 mm. Therefore, the modified PAP length can inform the user to determine a PAP, where the distance between the points defining the PAP, during motion of the joint, varies at least by the modified PAP length. That is, a PAP that varies by at least the modified PAP length should be determined to guide placement of the implant device 300. Depending on the laxity of the joint in a certain constrained degree of freedom, the PAP can be increased or reduced. In some embodiments, the PAP as a function of the laxity is defined by the manufacturer of the implant device 300. In some embodiments, the PAP as a function of the laxity is computed from a 3D biomechanical model of the patient's joint, including soft tissues that can alter the values of the laxity. In some embodiments, the PAP as a function of the laxity is computed from prior surgery data stored in a database, and where the surgical cases qualified by a surgeon as “successful” are averaged and then presented to the user during the surgery planification.

In a third step, the joint measurement data set, and specifically, data on joint kinematics can be gathered as described above and used to determine a plurality of PAPs that satisfy the modified PAP length. That is, a plurality of PAPs can be found, where the distance between each point defining each PAP varies by at least the modified PAP length. Satisfactory PAPs may be searched for only in the relevant anatomical area of the joint. For example, for a medial knee implant, PAPs will be computed on the medial surface of both the femur and the tibia. Based on the kinematics data, a plurality of PAPs can be determined that satisfy the modified PAP length, and which can therefore be used to determine or inform the placement of the implant device 300.

The PAPs can inform the placement of the implant device 300 on the joint. For instance, in some embodiments, the each support of the implant device 300 should be centered on a point of a selected PAP. As another example, in some embodiments, an anchor point of each support of the implant device 300 should be positioned on a point of a selected PAP. As another example, in some embodiments, the implant device 300 should be placed on the joint such that the linking member 308 extends linearly between the points defining the selected PAP when the joint is in a relaxed (e.g., straight) position. By extension, the placement of the supports on the joint can be determined based on the positioning of the linking member 308.

In a fourth step, the number of PAPs that satisfy the modified PAP length can be narrowed based on secondary considerations. For instance, if the supports of the implant device 300 were coupled to the joint in accordance with a particular PAP (as described in the above paragraph) it can be determined that the supports, anchor points, or fixation devices would violate a limiting zone (e.g., interfere with surrounding soft tissue or other bones). In which case, that particular PAP can be eliminated from the list of viable PAPs for guiding the placement of the implant device. Another secondary consideration can be the contact surface area between the supports and the first and second bones of the joint. For instance, if the supports of the implant device 300 were coupled to the joint in accordance with a particular PAP (as described in the above paragraph) the contact surface area between the supports and the first and second bones can be determined. A PAP resulting in a maximum contact surface area can be selected for guiding placement of the implant device 300. In some embodiments, a plurality of PAPs that would result in the contact surface area meeting a criteria can be selected for further consideration (e.g., PAPs that would result in the contact surface area not meeting the criteria can be discarded from consideration). It should be appreciated that this list is non-exhaustive, and that any of the factors used for determining the target implant parameters can be used to narrow the list of potential PAPs for guiding implant device 300 coupling to the joint (or for selecting the particular PAP for guiding coupling).

In some embodiments, if after implementing the secondary considerations, a plurality of PAPs still remain that can be used to guide the coupling the implant device 300 to the joint, a surgeon can select the particular PAP to use to guide implant device 300 coupling. For instance, the selection of the PAP can be done by the user in an interactive way. In some embodiments, the model of the implant device, along with the first and second bone models and optionally the models of the soft tissues can be displayed on a screen and the user can interact within the possible PAPs that guide implant device 300 placement until reaching a final decision. In some embodiments, the user selection can be stored in a database along with the joint measurement data set and later be used to feed a machine learning algorithm or a neural network which could be used to provide a PAP recommendation based on these previous data. In some embodiments, the final selection of the PAP can be made based on a biomechanical model of the first and second bone, along with the soft tissue, that computes the placement of the optimal device by optimizing the desired alignment, spacing or loading of the joint.

While the above examples were particularly discussed with respect to characterizing the PAP length with maximum Euclidean distance between points of a PAP, it should be appreciated that this is only an example. In some embodiments, the initial PAP length and modified PAP length can be traces of the variation of the Euclidean distance between each PAP as a function of the constrained or natural movement.

Also, while the above example discussed included modifying the PAP length based on the joint laxities, this is merely an example. In some embodiments, the operating parameter (e.g., stiffness, damping coefficient, tension, elasticity) of the linking member can be modified from an initial target operating parameter (e.g., set by the manufacturer or surgeon) based on the joint laxity data. Therefore, the joint kinematics data can be used to determine PAPs that satisfy the initial PAP length instead of a modified PAP length, since the PAP length is not modified based on joint laxity data.

It should be appreciated that the above method of using PAPs to determine the desired placement of the implant is one non-limiting example of determining the desired placement of the implant device.

It should be appreciated that the above list and methods is non-exhaustive, and that other factors can be considered to determine a desired placement of the implant device 300 to achieve the target joint parameters. Moreover, the above list of factors can be considered individually or in any combination to determine a desired placement of the implant device 300 to achieve the target joint parameters

Still referring to step 210, in some embodiments, the target implant parameter can include selecting a first support 302 or second support 304 of a desired shape and size to achieve the desired placement of the implant device 300 or to achieve the target joint parameter. For instance, the first support 302 and the second support 304 can come in variable sizes such that the first support 302 or second support 304 of a particular size and shape can be selected that is tailored to the patient's joint anatomy. In some examples, based on the 3D geometry of the first and second bone, along with the joint measurement data, the system might be able to suggest different implant devices sizes, if available, and the user will be able to choose one desired size.

Still referring to step 210, in some embodiments, the target implant parameters can include the desired placement of the one or more fixation devices used to couple the implant device 300 to the first bone or the second bone through the anchor points 306a,b. In some embodiments, the desired placement of the one or more fixation devices is determined based on one or more of the model (e.g., 3D model) of the implant device 300, the models (e.g., 3D model) of the first bone or the second bone, the characteristic points of the first bone or the second bone, the joint measurement data set (e.g., the joint laxities or joint kinematics), or the target joint parameters. The desired placement of the one or more fixation devices can be selected or determined to, at least in part, achieve the target joint parameters.

In some embodiment, the desired placement of the one or more fixation devices can be determined, at least in part, on the position of the one or more fixation devices on or in the first bone or the second bone. That is, in some embodiments, the desired placement of the one or more fixation devices can include a position of the fixation devices on the first bone or second bone. In some embodiment, the desired placement of the one or more fixation devices can include an orientation of the one or more fixation devices. That is, in some embodiments, the desired placement of the one or more fixation devices can include an orientation for the one or more fixation devices to be inserted into the first bone or the second bone. For instance, the orientation of the one or more fixation devices can be determined to maximize stress recovery. As an example, if the implant device 300 allows some degrees of freedom in the fixation device orientation, the determination can take into account these degrees of freedom to determine the desired fixation device orientation to have the best stress recovery. In some embodiment, the desired placement of the one or more fixation devices can be determined, at least in part, on the length, diameter, or other dimension, of the fixation devices. That is, in some embodiments, the desired placement of the one or more fixation devices can include the selection of fixation devices of known and desired dimensions. In some embodiment, the desired placement of the one or more fixation devices can be determined, at least in part, on a depth of insertion of the one or more fixation devices into the first bone or the second bone. For instance, the desired placement of the one or more fixation devices can include determining a desired depth of insertion of each of the one or more fixation devices. In some embodiments, the desired placement of the one or more fixation devices can be determined, at least in part, on the type (e.g., cancellous or cortical) or mechanical properties of the bone portions in which the fixation devices are to be placed. For example, the fixation device length can be determined to allow the fixation devices to be bi-cortical. It should be appreciated that the above list is non-exhaustive, and that other factors can be considered to determine a desired placement of the fixation devices to achieve the target joint parameters. Moreover, the above list of factors can be considered individually or in any combination to determine a desired placement of the fixation devices to achieve the target joint parameters.

Still referring to Step 210, in some embodiments, the target implant parameters can include a desired operating parameter of the linking member 308 of the implant device 300. The desired operating parameter can be the operating parameter for post-operatively achieving a desired alignment (e.g., a re-alignment of the joint or to maintain a pre-operative alignment of the joint) of the joint, a desired spacing of the joint, or a desired loading of the joint. In some embodiments, the desired operating parameter of the linking member 308 is determined based on one or more of the model (e.g., 3D model) of the implant device 300, the at least partial models (e.g., 3D models) of the first bone or the second bone, the characteristic points of the first bone or the second bone, the joint measurement data set (e.g., the joint kinematics or joint laxities), or the target joint parameters. The desired operating parameter of the linking member 308 can be selected or determined to, at least in part, achieve the target joint parameters. In some embodiments, the desired operating parameter of the linking member 308 can be determined through dynamic simulations of the positioning, movement, or loading of the first and second bones based on the operating parameter of the linking member 308. In some embodiments, the operating parameter of the linking member 308 can be determined such that the joint has a full range of flexion/extension and varus/valgus motion without device impingement. As discussed above, the operating parameter of the linking member 308 can be a characteristic affecting energy absorption of the linking member 308, such as the stiffness, tension, damping coefficient, or elasticity of the linking member 308. In some embodiments, the target operating parameter of the linking member 308 can be calculated based on the other target joint parameters such as alignment or gap values, and also based on the joint kinematics and/or laxities of the joint. For instance, a stiffer joint based on the laxities acquired in step 206 might require a stiffer implant device 300 to ensure a proper joint unloading. It should be appreciated that the above list is non-exhaustive, and that other factors can be considered to determine a desired operating parameter of the linking member 308 to achieve the target joint parameters. Moreover, the above list of factors can be considered individually or in any combination to determine a desired operating parameter of the linking member 308 to achieve the target joint parameters.

The placement of the implant device 300, the placement of the one or more fixation devices, and the operating parameter of the linking member 308 can be described as implant parameters herein. In some embodiments, the desired placement of the implant device, the desired placement of the fixation devices, and the desired operating parameter determined in step 210 can be determined pre-operatively. For instance, the desired placement of the implant device 300 can be determined prior to a surgical plan is confirmed and prior to the surgeon making an incision or other invasive step at the patient's joint. The implant parameters determined in step 210 can be referred to as initial implant parameters, which can be further refined, as discussed below.

In a step 212 of the method 200, in some embodiments, an initial surgical plan, including the determined desired placement of the implant device 300, desired placement of the fixation devices, or desired operating parameter of the linking member 308 can be presented. In some embodiments, the initial surgical plan can be presented on a user interface. In some embodiments, the user can validate or modify the initial surgical plan presented. For instance, if the surgeon is not satisfied with the proposed placement of the implant device 300, placement of the fixation devices, or the proposed operating parameter, the surgeon can modify such elements of the initial surgical plan. In some embodiments, the initial surgical plan can be presented to a user as a 3D model of the implant device 300 coupled to the first bone and the second bone. That is, the surgical plan can be presented to the user on an interface, where the presentation includes 3D models of the first bone, second bone, and implant device (showing for instance, where the implant device should be placed, where the fixation devices should be inserted into the bones, etc.).

In some embodiments, to determine implant parameters of the initial surgical plan in step 212 of the method 200, the localization of the first bone and the second bone in the constrained and natural positions (or ranges of motions) done in step 206 of the method 200, the comparison between the alignment or spacing of the joint in the relaxed position and alignment or spacing of the joint in the one or more constrained or natural positions (or ranges of motion), the determined distances between points in the constrained or natural positions (or ranges of motion) (e.g., joint profile, or PAP analysis), or the determined isometry pattern (which can be collectively referred to as the joint measurement data set) can be used. More specifically, a desired placement of the implant device 300, a desired placement of the one or more fixation devices, or a desired operating parameter of the linking member 308 can be determined based, at least in part, on the joint measurement data set. In some embodiments, the desired implant parameters can be determined based on the joint measurement data set and any or all of the factors discussed with respect to determining the desired implant parameters in step 210 above. In some embodiments, the joint measurement data set can be used to refine or modify the 3D model of the patient's joint or the biomechanical model of the patient's joint, discussed above, which can in turn be used, at least in part, to determine the desired implant parameters. The initial desired placement of the implant device 300, an initial desired placement of the one or more fixation devices, an initial desired operating parameter of the linking member 308 can enable for more precise and accurate attainment of the target joint parameter of the joint by accounting for the effects joint laxity, joint kinematics and surrounding soft tissue. In some embodiments, the initial surgical plan computed in step 212 of the method 200 can be executed and have computed holes positions of the anchoring points 306a,b of the implant device 300. In some embodiments the holes can be adapted to be screwed. In some embodiments, the holes can be adapted to receive Kirschner wires (K-wires) for a temporal placement.

Referring now to step 214 of the method 200, in some embodiments, a corrected surgical plan, including a corrected desired placement of the implant device 300, corrected desired placement of the one or more fixation devices, or corrected desired operating parameter of the linking member 308 can be presented.

In some embodiments, step 214 of the method 200 includes the surgical placement of a trial implant (e.g. an implant with the same dimensions as the implant 300 to be placed temporally only during surgery) with the sole purpose of testing the initial surgical plan and the initial implant parameters. For instance, the anchoring points 306a,b of the trial implant were computed previously in step 212. Once the trial implant is positioned using either K-wires or screws, the surgeon then can evaluate if the positioning of the trial implant matches the target joint parameters or one or more additional parameters. Such parameters can be described in the following non-exhaustive list: range of motion, minimum and maximum observed length or angle of the linking member 308, gap between the implant and the surrounding soft tissues such as ligaments or tendons, laxity observed with the implant. If one or more of the parameters achieved with the trial implant does not comply with the expected parameters, the surgeon can edit the initial surgical plan with corrected target implant parameters so as to meet the target joint parameters or one or more additional parameters described above. The modification and validation of the corrected plan can be presented as a 3D model with the first and second bone models and the implant device 300. The computer then calculates new variables to perform the surgery as modified in the corrected surgical procedure.

In some embodiments, step 214 of the method 200 requires the biomechanical model of the patient's joint (e.g. first and second bone and soft tissue) in order to simulate the desired position of the implant according to the step 212 using computational methods. In some embodiments, the computer simulation can be visualized as a 3D model that can be either manually or automatically moved in the constrained and natural degrees of freedom. In some embodiments, the computer simulation can produce as an output measured variables of the resulting movements of the joint with and without implant in order to compare and evaluate the implant positioning. Such variables can be present in the non-exhaustive list: joint angles, joint displacements, joint stiffness in one or more constrained degrees of freedom, range of motion, joint gap measurement, implant impingement, implant collision with surrounding tissues, joint loading, joint unloading, joint stress, joint strain.

In some embodiments, the corrected surgical plan can be presented on a user interface. In some embodiments, the user can validate or modify the corrected surgical plan presented. For instance, if the surgeon is not satisfied with the proposed placement of the implant device 300, placement of the fixation devices, or the proposed operating parameter, the surgeon can modify such elements of the corrected surgical plan. In some embodiments, the corrected surgical plan can be presented to a user as a 3D model of the implant device 300 coupled to the first bone and the second bone. The surgical plan presented in step 214, and the corrected desired position of the implant device 300, corrected desired position fixation devices, or corrected desired operating parameter of the linking member 308, can be particularly determined such that the target joint parameter of the joint is achieved post-operatively.

It should be appreciated, that in some embodiments, the method 200 need not present an initial surgical plan in step 212. For instance, in some embodiments, the first surgical plan presented to the user can be the surgical plan in step 214, which is based on the corrected desired placement of the implant device 300, corrected desired placement fixation devices, or corrected desired operating parameter of the linking member 308. Similarly, it should be appreciated that in some embodiments, the method 200 need not include determining initial implant parameters in step 210. For instance, the corrected implant parameters determined in step 214 can be the first implant parameters determined during the method 200. In other words, the implant parameters determined during step 214 do not need to be a correction of previously determined implant parameters. In such embodiments, it should be understood that the “corrected” desired implant parameters determined at step 214 can be based on the laxity, soft tissue or joint kinematic determinations made during step 206 and the further considerations discussed with respect to step 210 for determining the initial implant parameters. It should also be appreciated that, in some embodiments, no corrected surgical plan is determined in step 214. In such embodiments, the initial surgical plan determined and presented in step 212 is the final surgical plan presented to the user.

It should be appreciated that the method 200, and the particular steps thereof presented above, is merely one example workflow for determining implant parameters and a surgical plan to achieve desired joint parameters (e.g., a certain alignment, spacing, or loading of the joint) post-operatively. For instance, in some embodiments, the at least partial model of the first bone or the second bone can be obtained intraoperatively by tracking characteristic points of the first bone or the second bone with the localization system. A 3D model of the implant device, including its different possible sizes, can be known. Intraoperatively, the surgeon can manipulate the joint in the constrained positions, as discussed with respect to step 214, to determine an isometry map of the native joint. The surgeon can determine a desired post-operative joint parameter, including a desired isometry profile (which in some cases can match the isometry profile of the native joint, or can be an isometry profile to re-align the joint), for instance. Following this, the system can automatically determine, or determine with input from the surgeon, an initial surgical plan including, implant parameters determined to achieve the desired joint parameters post-operatively. In some embodiments, the surgeon can amend the initial surgical plan. Such amendments can be guided by the system which can alert or prohibit the surgeon from making impractical or incompatible amendments. The surgeon can validate the initial surgical plan or the amended surgical plan, which can then be carried out as discussed below.

The method 200, including its various determinations and logic discussed above, can be carried out by a control unit. The control unit can include at least one processor configured to implement algorithms to compute a joint parameter, a surgical plan, implant parameters, joint measurement data sets, bone models, and the like. The control unit can include at least one memory for storing data and executable instructions. The control unit can include a user interface to present information to a user and receive input from the user. In some embodiments, the user interface includes at least one display configured to display the surgical plan and, if applicable, modifications brought by the user.

For instance, in some embodiments, the control unit can, for instance, include one or more microprocessors, one or more random access memory (RAM) and/or one or more read-only memory (ROM), one or more calculators, one or more computers and/or one or more computer programs. The computer program(s) can include code instructions to determine the joint parameters, a surgical plan, implant parameters, joint measurement data sets, bone models, and the like. In addition, the control unit can include other devices and circuitry for performing the functions described herein such as, for example, a hard drive, input/output circuitry, and the like. The input/output circuitry can be adapted to treat digital and/or analog signals such as electromagnetic signals or video. In some embodiments, the control unit is configured to control or communicate with at least one input device, for receiving inputs and instructions from the surgeon and for presenting information, such as a surgical plan, to the surgeon.

Once the surgical plan presented in step 214 or 212 is finalized or validated by the surgeon, the surgical plan can be executed to re-align the joint. In some embodiments, the surgical plan can be implemented in a computer-assisted surgical procedure. In some embodiments, the surgical plan from the method 200 can be computed and executed simultaneously to re-align the joint.

Referring to FIG. 7, a surgical system 400 and environment for planning the surgical procedure, as discussed above, is depicted. In some embodiments, the patient lies on an operating table 402 in an operating room. A first tracker 404 is fixed to the patient's first bone and a second tracker 406 is fixed to the patient's second bone. In examples, the first tracker 404 is a tibial tracker fixed to the patient's tibia, and the second tracker 406 is a femoral tracker fixed to the patient's femur. The first tracker 404 and the second tracker 406 can be tracked by a localization system.

The localization system can employ any known localization technology. In some embodiments, the localization system can include at least one camera and the trackers can each include a respective set of markers with a unique geometry that can be detected by the camera. In some embodiments, the markers can be passive markers, such as disks or balls comprising a reflective surface adapted to reflect light from an infrared camera. In some embodiments, the passive markers can be polyhedral pins with visual markers inserted directly into the bodies to be tracked. In some embodiments, the markers can be active markers that are, themselves, adapted to emit light, such as LEDs. In some embodiments, markerless tracking can be used with the aid of 3D cameras segmenting and tracking, in real-time, the bodies to be tracked. In some embodiments, the tracking can be assured with tracked mini cameras that follows the motion of textures and other pattern characteristics of rigid tissues such as bones or cartilages. In some embodiments, the localization system can include at least one electromagnetic emitter that emits an electromagnetic field into the volume to be tracked, and the trackers can each include an electromagnetic sensors coupled to the localization system. In some embodiments, the localization system can include accelerometers, gyroscopes, and/or magnetometers. In some embodiments, the localization system can include IMU (Inertial Measurement Unit) sensors.

In some embodiments, a navigation station 408 can be positioned proximal to the operating table 402. The navigation station 408 can include a control unit 410 and a user interface 412. The user interface 412 can include a display, or other means for presenting information to the user. In some embodiments, the user interface 412 can include a touch screen or other input component for receiving information and commands from the user. The control unit 410 can include one or more processors configured to implement any of the above-described algorithms or logic.

The control unit 410 can be communicatively coupled to the localization system so as to receive localization data concerning the position of the trackers. In some embodiments, the control unit 410 can be a part of the localization system so as to receive localization data concerning the position of the sensors. For example, the first and second trackers 404, 406 can be electromagnetic trackers and belong to an electromagnetic localization system. The trackers 404, 406 can be communicatively linked to the control unit 410, via wired or wireless connection, to provide the control unit 410 with localization data. The trackers 404, 406 can be tracked in real time by the localization system to determine the position and orientation of the first bone and the second bone. The control unit 410, based on localization information, and the determined location of characteristic points relative to trackers 404, 406, for instance, can determine the position and orientation of characteristic points on the first and second bones.

In some embodiments, the surgical system also includes a 2D and/or 3D imaging system 414 that can be used pre-operatively or intra-operatively, as discussed above. The imaging system 414 can be brought next to the patient to acquire 2D and/or 3D images. For example, in some embodiments, an X-ray imaging system 414 can be a C-arm. The imaging system 414 can be communicatively coupled to the control unit 410 such that the control unit 410 can receive images acquired by the imaging system 414. Such a coupling allows the control unit 410 to place, in a common referential, the 2D or 3D images, the tracker localizations, and the characteristic points seen on the 2D or 3D images and tracked by the control unit 410.

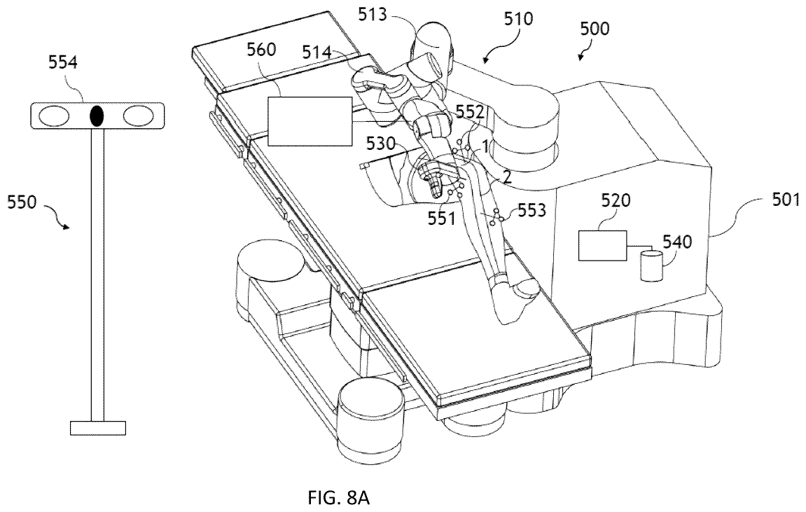

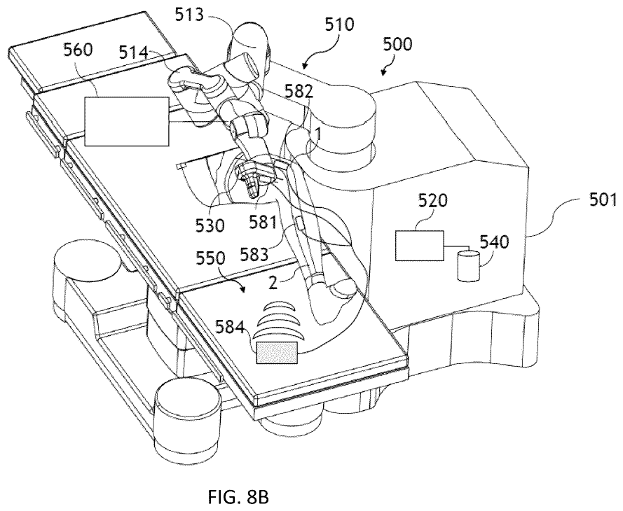

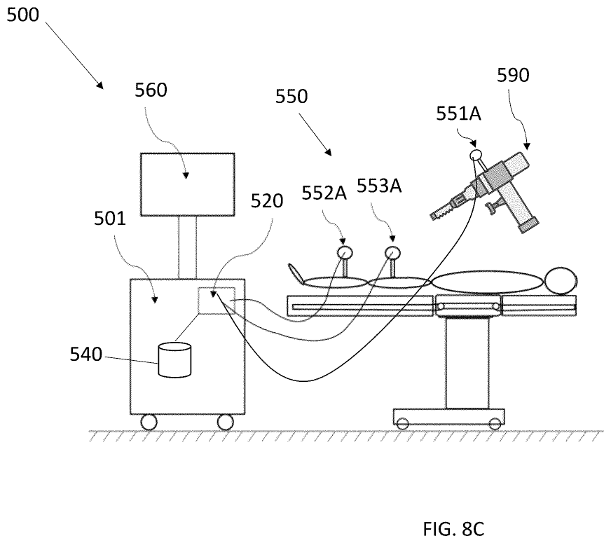

Referring now to FIGS. 8A, 8B, and 8C, a surgical system 500 for executing the surgical plan is depicted. It should be appreciated that like numerals in FIGS. 8A, 8B, and 8C can refer to the same elements. In some embodiments, the surgical plan can be implemented in a computer-assisted surgical procedure. In some embodiments, the hardware of the surgical system 500 can be incorporated into the surgical system 400. That is, a single surgical system can include the necessary hardware for planning and executing the surgical procedure. A common control unit, such as the control unit 410, can be coupled to and configured to monitor or control the various surgical planning and surgical execution hardware components.

The surgical system 500 can include a base 501 from which can, in some embodiments, extend a robotic arm 510. In some embodiments, the base 501 can be a wheeled cart adapted to be displaced in the operating room. The robotic arm 510 extends between a first end connected to the base 501 and a second end which forms a flange of the robotic arm 510. In some embodiments, the robotic arm 510 includes several segments 513 connected to one another thanks to motorized joints 514. In some embodiments, the robotic arm 510 can include at least six motorized joints 514. In some embodiments, the robotic arm 510 can include more or less than six motorized joints 514.

In some embodiments, one or more surgical tools 530 or passive tool guide 534 can be interchangeably attached to the flange of the robotic arm 510 to operate on the first bone or the second bone according to the surgical plan (e.g., according to the desired placement of the implant device 300 and/or the desired placement of the fixation devices). In some embodiments, in the case of the passive tool guide 534, the passive tool guide 534, itself, does not operate on the first bone or second bone, but is configured to only assist one or more additional devices to operate on the first bone or the second bone. In some embodiments, the robotic arm 510 constrains and precisely positions the one or more surgical tools 530 or passive tool guide 534. In some embodiments, at least one surgical tool 530 can be configured to drill fixation holes in the first bone and the second bone to receive the fixation devices. In some embodiments, at least one passive tool guide 534 can be placed to allow an external power tool to drill the fixation holes in the first and second bone to receive the fixation device. In some embodiments, the passive tool guide 534 can constrain the depth and the axis of the fixation holes drilled by the external power tool. By displacing the robotic arm 510, it is thus possible to drill a plurality of fixation holes according to a planned sequence.

In reference to FIG. 8C specifically, in some embodiments, the surgical system 500 can include an external surgical tool 590. In some embodiments, the surgical tool 590 can be a power tool. In some embodiments, the surgical tool 590 can be a power drill. In some embodiments, the surgical tool 590 can be manually controlled and maneuvered. In such embodiments, the surgical system 500 may not include a robotic arm 510.

In some embodiments, the surgical system 500 includes a control unit 520 (or the control unit 410, as noted above). In some embodiments, such as those shown in FIGS. 8A and 8B, the control unit 520 is configured to control the robotic arm 510. In some embodiments, the control unit 520 can, for instance, include one or more microprocessors, one or more random access memory (RAM) and/or one or more read-only memory (ROM), one or more calculators, one or more computers and/or one or more computer programs. The computer program(s) can include code instructions to control the robotic arm 510. In some embodiments, as shown in FIG. 8C, the computer program(s) can include code instructions to provide surgical instructions to a human user to maneuver and control the surgical tool 590. In addition, the control unit 520 can include other devices and circuitry for performing the functions described herein such as, for example, a hard drive, input/output circuitry, and the like. The input/output circuitry can be adapted to treat digital and/or analog signals such as electromagnetic signals or video.

The surgical system 500 can include at least one input device (such as the user interface 412 or user interface 560) and at least one processing unit adapted to receive inputs transmitted by the user through the input device and to compute instructions to be sent to one or more motorized joints 514 of the robotic arm 510 so as to displace the surgical tool 530 or the passive tool guide 534 and to activate the surgical tool 530 as commanded by the user, but with safety parameters—not detailed in this document—considered by said processing unit. For instance, in some embodiments, the input device can be formed as a touchscreen, a joystick or a handle mounted on the robotic arm 510, near the surgical tool 530 or the passive tool guide 534. The processing unit can be integrated in the control unit 520 or configured to communicate with the control unit 520.

In some embodiments, the surgical system 500 includes a tracking system 550. In some embodiments, the tracking system 550 can be configured to determine, at a predetermined frequency, for example greater than 100 Hz, a relative pose of the robotic arm 510, or the surgical tool 590, with respect to the first and the second bone, and to send corresponding localization data to the control unit 520. To that end, the tracking system 550 can include at least a first tracker attached to the robotic arm 510, or the surgical tool 590, a second and a third tracker respectively fixed to the first and second bones (as discussed above). In some embodiments, the first tracker can be attached to the flange of the robotic arm 510. In some embodiments, the first tracker can be arranged on any segment of the robotic arm 510. In some embodiments, the first tracker can be arranged on the base 501 of the surgical system 500. In some embodiments, a kinematic model of the robotic arm 510 is stored in a memory of the control unit 520, and the control unit 520 is thus able to determine the pose of the surgical tool 530 or the passive tool guide 534 relative to the first tracker based on the kinematic model.

In some embodiments, the first and second trackers can be fixed respectively to the first and second bones by screwing or impacting.

Referring to FIG. 8A, in some embodiments, the tracking system 550 can be an optical tracking system. The tracking system 550 can include a first tracker 551 rigidly fixed to the robotic arm 510, a second and third trackers 552, 553 rigidly attached respectively to the first and second bones 1, 2 and a camera system 554 configured to detect the current pose of the first, second and third trackers 551, 552, 553 and to determine, based on the current poses of the trackers 551, 552, 553, the relative pose (e.g., position and orientation) of the robotic arm 510 (and the surgical tool 530 and/or the passive tool guide 534) with respect to the first and second bone 1, 2. In some embodiments, the tracking system 550 referred as the optical tracking system can consist in a camera 554 attached to the surgical instrument such as the drill tracking directly the fixed trackers 552 and 553 attached to the first and second bones in order to determine the current pose of the instrument relative to any of the bones. It should be appreciated that this is a non-limiting example of optical tracking systems that can be employed.

Referring to FIG. 8B, in some embodiments, the tracking system 550 can be an electromagnetic tracking system. The electromagnetic tracking system can include at least a first, second and third trackers 581, 582, 583 configured to receive an electromagnetic field, and an emitting device 584 configured to emit the electromagnetic field. The tracking system 550 is configured to determine, from the electromagnetic field received by the trackers 581, 582, 583, the relative pose of the robotic arm 510 (and the surgical tool 530 or the passive tool guide 534) with respect to the first and second bones 1, 2.

Referring to FIG. 8C, in some embodiments, the tracking system 550 can be any of the above-described tracking or localization systems. The tracking system 550 can include at least a first tracker 551A rigidly fixed to the surgical tool 590, a second and third trackers 552A, 553A rigidly attached respectively to the first and second bones 1, 2 and a system (e.g., a camera system) configured to detect the current pose of the first, second and third trackers 551A, 552A, 553A and to determine, based on the current poses of the trackers 551A, 552A, 553A, the relative pose (e.g., position and orientation) of the surgical tool 590 with respect to the first and second bone 1, 2.

In some embodiments, the surgical system 500 includes a storage unit 540, the control unit 520 being configured to communicate with the storage unit 540. This communication can be wired or wireless without departing from the scope of the disclosure. In some embodiments, as shown, the control unit 520 and the storage unit 540 can be embedded within the base 501. It should be appreciated that the control unit 520 and storage unit 540 can be positioned anywhere else within the system 500 or remote from the system 500. The storage unit 540 can be configured to store a planned surgical procedure. The storage unit 540 can be configured to store a 3D model of the first bone 1 and the second bone 2. The 3D model can be the 3D model discussed above with respect to the method 200. The 3D model of the first bone 1 and the second bone 2 can include the desired placements of the implant device 300 and the fixation devices. In some embodiments, the 3D model of the first bone 1 and the second bone 2 can include a representation of the desired operating parameter of the linking member 308. Therefore, in some embodiments, the 3D model includes a placement of the implant device 300 and of the plurality of fixation devices used to fix the implant device 300 to the first bone 1 and the second bone 2.

In some embodiments, the control unit 520 is configured to compute the pose of each drilling axis corresponding to the desired placement of the implant device 300 (and its anchor points 306a,b) and the desired placement of the fixation devices included in the planned surgical procedure. In some embodiments, it should be appreciated that the drilling axis corresponding to the desired placement of the implant device 300 (and its anchor points 306a,b) and the desired placement of the fixation devices, can themselves be included in the planned surgical procedure.

Referring to FIG. 8C, in particular, in some embodiments, the tracking system 550 can be in communication with the controller 520. The controller 520 can be configured to present, on the user interface 560, visualizations of the pose, position, or orientation of the first and second bones 1,2 in real-time. In some embodiments, the visualizations can be 3D models of the first bone 1 and the second bone 2. In some embodiments, the controller 520 can be configured to present, on the user interface 560, a visualization of the pose, position, or orientation of the surgical tool 590 in real-time. In some embodiments, the controller 520 can be configured to present, on the user interface 560, visualizations of the poses, positions, or orientations of the first bone 1, the second bone 2, and the surgical tool 590, simultaneously, in real-time. The 3D model of the first bone 1 and the second bone 2 can include the corrected desired placements of the implant device 300 and the fixation devices. Therefore, in some embodiments, the 3D model includes a placement of the implant device 300 and of the plurality of fixation devices used to fix the implant device 300 to the first bone 1 and the second bone 2. In some embodiments, the 3D model of the first bone 1 and the second bone 2 can include a representation of the corrected desired operating parameter of the linking member 308.

In some embodiments, the user or surgeon can freely manipulate the surgical tool 590. The visualization presented on the user interface 560 can aid the surgeon in properly positioning the surgical tool 590 relative the holes or drilling axes for the fixation devices in the first bone 1 and the second bone 2. In some embodiments, the controller 520 can present, on the user interface 560, a suggestion for the surgeon to manipulate the surgical tool 590. For instance, the user interface 560 can present a suggestion that the surgeon should move the surgical tool 590 5 cm in the medial direction to align the surgical tool 590 with a drilling axis.

The surgical tool 590 can be any surgical tool for carrying out the procedure. In some embodiments, the surgical tool 590 is a power drill. In some embodiments, the surgical tool 590 includes a drill bit. In some embodiments, the power source for the surgical tool 590 can be electrical (battery powered or with an electrical cord), pneumatic or hydraulic. In some embodiments, the power tool 590 can be a handheld robotic power tool linked by wire or wirelessly to the control unit 520 which controls and rectifies the target aim dynamically of the tool tip based on the surgical plan transmitted by the control unit 520. That is, while some embodiments below may refer to controlling a robotic arm to position the surgical instrument, in some embodiments, the surgical tool 590 can be a handheld robotic tool that dynamically assists the user in properly positioning the surgical tool for performing the surgery (e.g., drilling the necessary holes for coupling the implant device to the bone).

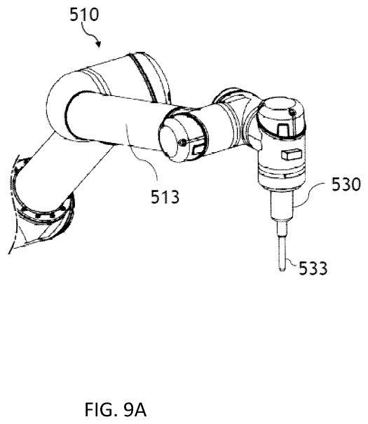

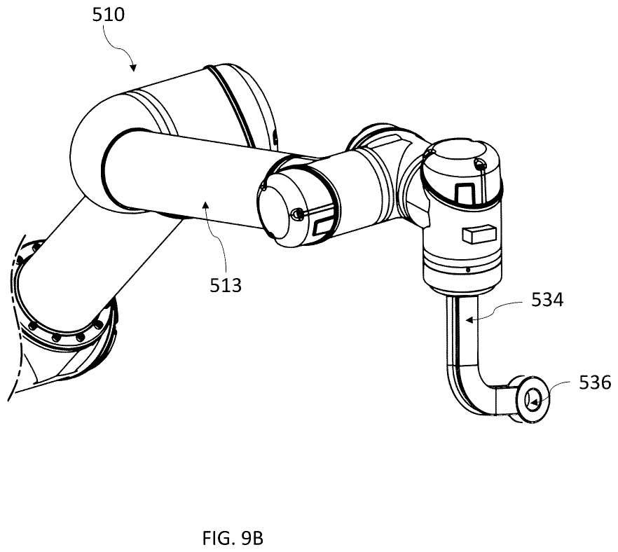

Referring more specifically to FIGS. 8A and 8B, in some embodiments, the control unit 520 unit can be configured to control the robotic arm 510 to align the surgical tool 530 with the determined drilling axes based on the localization data received from the tracking system. By “aligning,” it is meant that an active portion of the surgical tool 530 is within the drilling axis. “Active portion” refers to the portion of the surgical tool 530 that is configured to drill the region of interest of the first bone 1 and the second bone 2. In the example illustrated in FIG. 9A, the surgical tool 530 is a drill, and the active portion of the drill 530 is formed by the surface 533 of the drill bit. The control unit 520 is configured to compute movement(s) and send instructions to control one or several motorized joints 514 of the robotic arm 510 to align the surgical tool 530 with the determined drilling axis based on the localization data.

In some embodiments, the control unit 520 unit can be configured to control the robotic arm 510 to align the passive tool guide 534 with the determined drilling axes based on the localization data received from the tracking system. In some embodiments, by “aligning,” it is meant that the guiding portion of the passive tool guide 534 is within the drilling axis. In some embodiments, “guiding portion” refers to the portion of the passive tool guide 534 that is configured to guide an external surgical tool to drill the region of interest of the first bone 1 and the second bone 2. In the example illustrated in FIG. 9B, the passive tool guide 534 includes a surface 536 that is configured to receive an external surgical tool, such as a driller, such that the external surgical tool, and particularly the active portion of the surgical tool, is aligned with the drilling axes. In other words, the surface 536 can be aligned with the drilling axes, and a surgeon can then place the external surgical tool in, on, or through the surface 536 such that the surgical tool, and particularly the active portion of the surgical tool is aligned with the drilling axes. The control unit 520 is configured to compute movement(s) and send instructions to control one or several motorized joints 514 of the robotic arm 510 to align the passive tool guide 534, and therefore the surgical tool received by the passive tool guide 534, with the determined drilling axis based on the localization data.

In some embodiments, the alignment of the surgical tool 530 or the passive tool guide 534 can be done automatically by the robotic arm 510 based on the planning and on localization data provided by the tracking system. In some embodiments, the alignment of the surgical tool 530 or the passive tool guide 534 can be instructed by the user via the input device. The control unit 520 can process the instructions and provide commands to the motorized joints 514 based on the user's input, the surgical planning, and the localization data provided by the tracking system.

In some embodiments, in order to allow the user of the system to operate the surgical tool 530 safely, the control unit 520 can be further configured to allow an activation of the surgical tool 530 only when the surgical tool 530 is aligned with the drilling axis. In some embodiments, the surgical tool 530 can be activated or guided by the user, through the input device. In some embodiments, the control unit 520 can be configured to activate the surgical tool 530 to drill the bones automatically, based on the surgical planning and on the determined poses of the concerned bones obtained via the tracking system.

In some embodiments, the passive tool guide 534 can be positioned or guided by the user through the input device. In some embodiments, the passive tool guide 534 is positioned so that when the external surgical tool is received in the passive tool guide 534, the main axis of action of the external surgical tool is the axis target in which the user can manually activate the external surgical tool to drill. In some embodiments, the control unit 520 can be configured with the size of the drilling bit and the target drilling depth (which can then stored in the storage unit 540) so that the control unit 520 can automatically position the passive tool guide 534, and particularly the surface 536, such that the external surgical tool reaches the target drilling depth when operated. In some embodiments, for the case of multiple drill holes, the control unit 520 can receive a user input once a first drilled hole is complete, and after receiving the input, the control unit 520 can automatically move the passive tool guide 534 to a new position to drill a subsequent hole.

The control unit 520 can be configured to drill a fixation device hole into the first bone 1 and the second bone 2 at the desired position, angle, and depth determined for the planned surgical procedure. Therefore, the control unit 520 can be configured to displace the surgical tool 530 along the drilling axis until reaching the predetermined depth for a respective fixation device. Consequently, the surgical system 500 does not drill further than a “virtual end limit” (i.e., the predetermined depth for the fixation device) which prevents over-drilling risks such as drilling soft tissues. In some embodiments, each predetermined drilling axis (including position and angle) and predetermined drilling depth can be stored in the storage unit 540.

Various modes of visual representation known in the art can be employed on the user interface 560 to present any of the above-described visual aides. In some embodiments, the user interface 560 can be an LCD screen. In some embodiments, the user interface 560 can present an augmented reality representation by means of virtual reality glasses or a transparent display screen.

It should be clear from the above description that, in some embodiments, the surgical system 500 can be configured to display, in real-time, models of the implant device, first bone, second bone, surgical instrument (manually or automatically operated), or tool guide relative to each other. That is, the system can determine the pose of at least some of the first bone, second bone, implant device, surgical instrument, or tool guide relative each other and display such models according to the real-time poses. In some embodiments, the models of the first or second bones can be annotated with the poses of the fixation device holes to be drilled according to the surgical plan. Based on the relative poses, the system can instruct a robotic arm, for instance, to move an instrument relative the first or second bone to execute the surgical plan (e.g., drill fixation device holes). Based on the relative poses, the system can instruct a robotic arm, for instance, to move a passive tool guide relative the first or second bone to execute the surgical plan (e.g., position the tool guide such that a tool aided by the tool guide can drill fixation device holes). Based on the relative poses, the system can instruct a handheld robotic device to move itself to assist a user in manual manipulation of the handheld robotic device to execute the surgical plan (e.g., drill fixation device holes). Based on the display presented by the system, a user can be move surgical instrument relative the first bone and second bone to execute the surgical plan (e.g., drill fixation device holes).

In some embodiments, the surgical system 500 can be configured to allow the surgeon to select easily and quickly a proper fixation device among a lot of fixation device references. To do that, in some embodiments, the storage unit 540 can store a plurality of fixation device references, such as a fixation device database, each fixation device reference defining a type (e.g. mono-cortical screw or bi-cortical screw), a diameter, and a length of a respective fixation device. The control unit 520 can be further configured to select, for the fixation device hole, a fixation device reference whose diameter is less than or equal to a diameter of the fixation device hole and whose length corresponds to the predetermined depth of the fixation device hole. In such embodiments, it is therefore not necessary for the user to test, and thus waste, several fixation devices in the fixation device hole until finding a proper fixation device. In some embodiments, the fixation can be either permanent (such as screws) or temporary (such as Kirschner wires).

In some embodiments, the control unit 520 can be further configured to determine a maximum or a minimum relative speed of displacement of the surgical tool 530, or passive tool guide 534, or the surgical tool 590 with respect to the predetermined drilling axis based on the localization data provided by the tracking system. In some embodiments, a plurality of maximum or minimum relative speeds may be determined so as to adapt the relative speed of the surgical tool 530 as required by the surgeon. For example, when the surgical tool 530 or passive tool guide 534 is remote to the drilling axis, the robotic arm 510 may need to displace the surgical tool 530 quickly or passive tool guide 534 quickly, and when the surgical tool 530 or passive tool guide 534 is close to the drilling axis, the robotic arm 510 may need more accuracy and therefore may need to displace the surgical tool 530 or passive tool guide 534 at a lower speed but also above a minimum speed to drill accurately. In some embodiments, the minimum relative speed is zero. In some embodiments, the control unit 520 can compute instructions to control the surgical tool 530 or passive tool guide 534 based on the maximum or the minimum relative speed previously determined. In some embodiments, the control unit 520 can present a recommendation to a surgeon as to how fast he or she should be manipulating the surgical tool 590 toward, in, or through the first bone 1 or the second bone 2.

In some embodiments, the control unit 520 can be configured to determine a maximum or a minimum rotation speed of the surgical tool 530 based on the localization data provided by the tracking system. The rotation speed can be the rotation of a drill. The maximum or minimum rotation and relative speeds allow the user to drill without damaging the bones, for example by overheating bone tissues or by vibrations. Of course, a plurality of maximum or minimum rotation speeds can be determined so as to adapt the rotation speed of the surgical tool 530 as required by the surgeon. Indeed, cancellous and cortical bones do not have the same hardness. In addition, cancellous bone does not overheat at the same temperature as the cortical bone. Therefore, determining different maximum or minimum rotation speeds allows to improve accuracy when drilling is performed. In some embodiments, the minimum rotation speed is zero. In some embodiments, the control unit 520 can present a suggestion to a surgeon for a rotational speed of the surgical tool 590 to drill a hole for a fixation device.

In some embodiments, the control unit 520 can compute instructions to control the surgical tool 530 based the maximum or the minimum rotation speed previously determined. The control unit 520 can be further configured to determine the relative speed and the rotation speed based on a size of the surgical tool 530, for example a length of the surgical tool 530, and a design of the surgical system 500, for example specifications of the motorized joints 514.

In some embodiments, the surgical system 500 includes the at least one user interface 560, which can be configured to display a representation computed by the control unit 520 of the pose of the surgical tool 530, passive tool guide 534, or the surgical tool 590 with respect to each predetermined drilling axis, a representation of the predetermined drilling axis, a representation of the predetermined depth, or the 3D model of the first and second bones, the implant device, the surgical tool 530, passive tool guide 534, or the surgical tool 590. Thus, the user of the system can appreciate at any time the relative pose of the above objects with respect to one another. In some embodiments, the user interface 560 can be further configured to display, for each fixation device hole, a representation of the selected fixation device reference. Thus, the user can select a proper fixation device quickly. According to the illustrated embodiment, the user interface 560 is formed as a display attached to the robotic arm 510. Obviously, this is merely an example and the display could be positioned on another part of the surgical system 500, such as its base, within the scope of the disclosure. Indeed, in the embodiment depicted in FIG. 8C, no robotic arm exists. In some embodiments, the user interface 560 can be attached to the operating table. In some embodiments, the user interface 560 can integrate a touch-sensitive interface to receive inputs transmitted by the user. In some embodiments, the user interface 560 can be remotely controlled by the control unit 520. In some embodiments, the user interface 560 can be an augmented reality device configured to be positioned between the eyes of the user and the region of interest, such as augmented reality glasses, goggles, or panel.

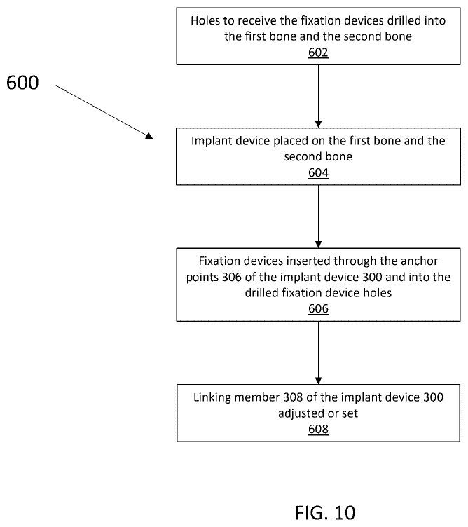

Referring now to FIG. 10, a method 600 of performing the planned surgical procedure featuring the desired implant parameters is depicted. The method 600 can be performed by the surgical system 500 based on the finalized planned surgical procedure discussed with respect to the method 200 of FIG. 2. In some embodiments, in step 602, holes to receive the fixation devices can be drilled into the first bone and the second bone. The holes can be drilled according to the principles discussed with respect to FIGS. 8 and 9, and particularly in accordance with the target implant parameters, such as desired placement of the fixation devices determined during the method 200. That is, the fixation devices holes can be drilled at a position, orientation, or dimensions (such as depth) in accordance with the desired fixation device parameters. The fixation device holes can, also be drilled in accordance with the desired placement of the implant device 300 determined during the method 200.

In step 604, in some embodiments, the implant device can be desirably placed on the first bone and the second bone in a certain joint kinematic configuration (for example a certain flexion or abduction angle) according to the planning from the method 200. Specifically, the first support 302 can be desirably placed on the first bone 1, and the second support 304 can be desirably placed on the second bone 2. The implant device 300 can be desirably placed on the first bone 1 and the second bone 2 in accordance with the desired implant parameters, such as placement, of the implant device 300 determined during the method 200. The implant device 300 can be desirably placed on the first bone 1 and the second bone 2 such that the anchor points 306a of the first support 302 overlap or align with the fixation device holes drilled in the first bone 1, and the anchor points 306b of the second support 304 overlap or align with the fixation device holes in the second bone 2.

In step 606, in some embodiments, the fixation devices can be inserted through the anchor points 306a,b of the implant device 300 and into the drilled fixation device holes to couple the implant device 300 to the first bone 1 and the second bone 2. The implant device 300 can be coupled to the first bone 1 and the second bone 2 in accordance with the target implant parameters, including desired placement of the implant device 300 and the desired placement of the fixation devices determined during the method 200.

In step 608, in some embodiments, the linking member 308 of the implant device can be adjusted or set in accordance with the target implant parameters, such as desired operating parameter, determined during the method 200. In some embodiments, the linking member 308 is joined together with the first and second support 302, 304, and therefore can only be adjusted depending on the joint kinematic configuration, either with a natural degree of freedom (for example, an angle of flexion or abduction) or with a constrained degree of freedom (for example, a varus angle or any other degree of freedom related to the laxity). In some embodiments, a user can manually set or adjust the operating parameter, such as a tension, of the linking member 308. In some embodiments, a robotic arm can be guided to set or adjust the operating parameter of the linking member 308. In some embodiments, the implant device 300 can be compatible with a plurality of linking members 308. That is, different linking members 308 can be coupled to and extend between the first support 302 and the second support 304 of the implant device 300. In such embodiments, a user can manually attach a linking member 308 configured with or possessing the corrected desired operating parameter to the implant device 300. In some embodiments, a robotic arm can be guided to attach a linking member 308 configured with or possessing the corrected desired operating parameter to the implant device 300.

It should be appreciated that the methods discussed above need not be completed in the order of steps discussed above. Multiple steps of the methods can be performed substantially simultaneously or in a different order than presented above. Similarly, multiple steps of the methods can be omitted, or additional steps could be added. For instance and with respect to the method 600 specifically, in some embodiments, the implant device 300 can first be placed on the first bone 1 and the second bone 2 in accordance with the target implant parameters, including desired placement of the implant device 300. Subsequently, the fixation device holes can be drilled through the anchor points 306a,b of the implant device 300 and into the first bone 1 and the second bone 2. As another example, it should be appreciated that the linking member 308 can be adjusted or set in accordance with the target implant parameters, including the desired operating parameter before the implant device 300 is coupled to the first bone 1 and the second bone 2.

Techniques operating according to the principles described herein may be implemented in any suitable manner. Included in the discussion above are flow charts showing the steps and acts of various processes that determine a surgical plan for implanting an implant device on a joint and execute a surgical plan for implanting the implant device. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit, Field Programmable Gate Array (FPGA), or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one of ordinary skill in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of software. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

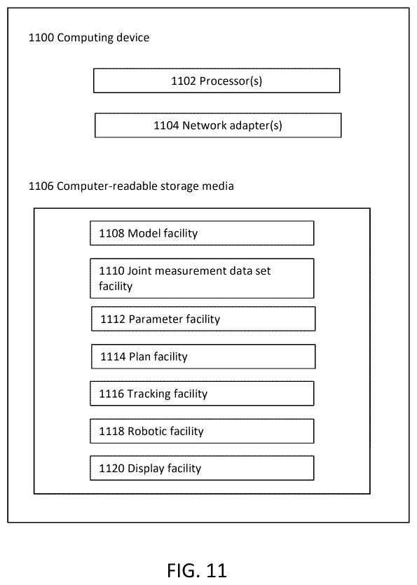

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 1106 of FIG. 11 described below (i.e., as a portion of a computing device 1100) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system or one or more computing devices (or one or more processors of one or more computing devices) may be programmed to execute the computer-executable instructions. A computing device or processor may be programmed to execute instructions when the instructions are stored in a manner accessible to the computing device/processor, such as in a local memory (e.g., an on-chip cache or instruction register, a computer-readable storage medium accessible via a bus, a computer-readable storage medium accessible via one or more networks and accessible by the device/processor, etc.). Functional facilities that comprise these computer-executable instructions may be integrated with and direct the operation of a single multi-purpose programmable digital computer apparatus, a coordinated system of two or more multi-purpose computer apparatuses sharing processing power and jointly carrying out the techniques described herein, a single computer apparatus or coordinated system of computer apparatuses (co-located or geographically distributed) dedicated to executing the techniques described herein, one or more Field-Programmable Gate Arrays (FPGAs) for carrying out the techniques described herein, or any other suitable system.

FIG. 11 is a block diagram of an example computing system for determining a surgical plan for implanting an implant device on a joint and executing a surgical plan for implanting the implant device. FIG. 11 illustrates one possible implementation of a computing device in the form of a computing device 1100 that may be used in a system implementing techniques described herein, although others are possible. It should be appreciated that FIG. 11 is intended neither to be a depiction of necessary components for a computing device to determine a surgical plan for implanting an implant device on a joint and execute a surgical plan for implanting the implant device, nor a comprehensive depiction.

Computing device 1100 may include at least one processor 1102 (e.g., a computer hardware processor), a network adapter 1104, and computer-readable storage media 1106. Computing device 1100 may be, for example, a desktop or laptop personal computer, a personal digital assistant (PDA), a smart mobile phone, or any other suitable computing device. Network adapter 1104 may be any suitable hardware and/or software to enable the computing device 1100 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable storage media 1106 may be adapted to store data to be processed and/or instructions to be executed by processor 1102. Processor 1102 enables processing of data and execution of instructions. The data and instructions may be stored on the computer-readable storage media 1106.

The data and instructions stored on computer-readable storage media 1106 may comprise computer-executable instructions implementing techniques which operate according to the principles described herein. In the example of FIG. 11, computer-readable storage media 1106 stores computer-executable instructions implementing various facilities and storing various information as described above. Computer-readable storage media 1106 may store model facility 1108 configured to perform techniques described herein, such as determining models of the implant device or determining models of the first and second bone of the joint. Computer-readable storage media 1106 may store joint measurement data set facility 1110 configured to perform techniques described herein, such as gathering a joint measurement data set, which can include kinematic or laxity data. Computer-readable storage media 1106 may store parameter facility 1112 configured to perform techniques described herein, such as determining target joint parameters or determining target implant parameters. Computer-readable storage media 1106 may plan facility 1114 configured to perform techniques described herein, such as determining a surgical plan. Computer-readable storage media 1106 may store tracking facility 1116 configured to perform techniques described herein, such as tracking the first bone and the second bone, tracking a robotic arm, tracking a surgical tool, and the like. Computer-readable storage media 1106 may store robotic facility 1116 configured to perform techniques described herein, such as controlling a robotic arm, passive tool guide, or hand held robot to execute or assist a surgical plan. Computer-readable storage media 1106 may store display facility 1116 configured to perform techniques described herein, such displaying models or a surgical plan to the user on a display and receiving user feedback.

While not illustrated in FIG. 11, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments

Based on the foregoing, it should now be understood that embodiments shown and described herein relate to systems and method for guiding use of surgical implants having a linking member.

The present disclosure relates to a system, including: a localization system, the localization system including a detector and one or more trackers, wherein: the detector is configured to localize the one or more trackers; and the localization system is configured to generate a joint measurement data set of a joint, wherein the joint is defined by a first bone and a second bone; and a control unit including a processing device configured to: receive the joint measurement data set from the localization system; based on the joint measurement data set, determine a surgical plan for a surgery, wherein: the surgery includes fixing an implant device to the joint; the surgery is configured to be carried out either by a robotic system or only with a navigated external power tool; the implant device includes: a first support configured to be fixed to the first bone; a second support configured to be fixed to the second bone; and a linking member extending between the first support and the second support; and the surgical plan includes one or more implant parameters of the implant device, wherein the implant parameters include: a placement of the first support on the first bone; a placement of the second support on the second bone; and an operating parameter of the linking member.

The present disclosure relates to a system, including: an implant device, including: a first support configured to be fixed to a first bone; a second support configured to be fixed to a second bone; and a linking member extending between the first support and the second support; and a robotic system, including: a robotic arm configured to hold one or more surgical tools; and a processor configured to: determine a surgical plan for a surgery on a joint including the first bone and the second bone, wherein the surgical plan includes: a placement of the first support on the first bone; a placement of the second support on the second bone; and an operating parameter of the linking member; and operate the robotic arm and the one or more surgical tools to: drill one or more holes into the first bone and the second bone for fixing the first support to the first bone and the second support into the second bone, respectively; and set the operating parameter of the linking member.

The present disclosure relates to a system, including: an implant device, including: a first support configured to be fixed to a first bone; a second support configured to be fixed to a second bone; and a linking member extending between the first support and the second support; a processor configured to: determine a surgical plan for a surgery on a joint including the first bone and the second bone, wherein the surgical plan includes: a placement of the first support on the first bone; a placement of the second support on the second bone; and an operating parameter of the linking member; and a robotic system, including: a robotic arm configured to hold one or more passive tool guides, wherein the passive tool guide is aligned to each of the plurality of drilling axes according to the surgical plan for the fixation of the first support and the second support of the implant device; and an external power tool that is a surgical driller with interchangeable drilling bits, wherein the drilling bits are aligned with the passive tool guide according to the plurality of drilling axes of the surgical plan.

The present disclosure relates to a system, including: a localization system including a detector and one or more trackers, wherein: the detector is configured to localize the one or more trackers wherein: a first tracker is attached to a first bone, a second tracker is attached to a second bone, the two bones making a joint, and a third tracker is attached to an external power tool to perform surgical actions; the external power tool is a surgical driller with interchangeable drilling bits; and a processor in communication with the localization system, the processor configured to: show, on a display, the position and orientation in real-time of the first and second bone with the attached trackers and the external power tool with the attached tracker; and determine a surgical plan for a surgery on the joint including the first bone and the second bone, wherein the surgical plan includes: the plurality of drilling axes to be drilled by the external power tool with the expected predetermined depth.

The present disclosure relates to a system, including a control unit; a robotic arm, the control unit being configured to control the robotic arm; a surgical tool configured to be attached to the robotic arm and to drill at least one fixation device hole in a first bone or a second bone along a respective predetermined drilling axis and until a respective predetermined depth, wherein the first bone and the second bone define a joint; and a localization system including a first tracker configured to be fixed to the robotic arm, and a second tracker and a third tracker configured to be fixed, respectively, to the first bone and the second bone, wherein the localization system is configured to determine a relative pose of the robotic arm with respect to the first bone and the second bone, and to send corresponding localization data to the control unit, wherein the control unit is configured to: compute the respective drilling axis and the respective predetermined depth based on a planned position of an implant device on the first bone and the second bone and based on a planned pose of a plurality of fixation devices configured to secure the implant device to the first bone and the second bone, wherein the implant device includes: a first support configured to be fixed to the first bone; a second support configured to be fixed to the second bone; and a linking member extending between the first support and the second support; and control the robotic arm to: align the surgical tool with the predetermined drilling axis based on the localization data provided by the localization system; and displace the surgical tool along the predetermined drilling axis until reaching the predetermined depth based on the localization data.

The present disclosure relates to a method, including: collecting a joint measurement data set for a joint, wherein the joint is defined by a first bone and a second bone; and based on the joint measurement data set, determining a surgical plan for a surgery, wherein: the surgery includes fixing an implant device to the joint; the implant device includes: a first support configured to be fixed to the first bone; a second support configured to be fixed to the second bone; and a linking member extending between the first support and the second support; and the surgical plan includes one or more implant parameters of the implant device, wherein the implant parameters include: a placement of the first support on the first bone; a placement of the second support on the second bone; and an operating parameter of the linking member.

The present disclosure relates to a method, including: providing an implant device, wherein the implant device includes: a first support configured to be fixed to a first bone; a second support configured to be fixed to a second bone; and a linking member extending between the first support and the second support; determining a surgical plan for a surgery on a joint including the first bone and the second bone, wherein the surgical plan includes: a placement of the first support on the first bone; a placement of the second support on the second bone; and an operating parameter of the linking member; drilling one or more holes, by means of a robotic surgical tool, by a external power tool guided by a passive tool guide, or by a navigated external power tool, into the first bone and the second bone for fixing the first support to the first bone and the second support into the second bone, respectively; and setting the operating parameter of the linking member.

In some embodiments, the joint measurement data set includes localization data of one or more characteristic points of at least one of the first bone or the second bone when the joint is in one or more constrained positions.

In some embodiments, the systems and the methods further include applying one or more forces to the joint to position the joint in the one or more constrained positions.

In some embodiments, each of the one or more constrained positions are selected to simulate the effects of laxities and soft tissues on the alignment of the joint.

In some embodiments, the localization data of the one or more characteristic points is determined with a localization system configured to localize one or more trackers coupled to at least one of the first bone or the second bone.

In some embodiments, the systems and methods further include, based on the joint measurement data set, determining a biomechanical model of the joint, wherein: the biomechanical model accounts for the effects of laxities and soft tissues on the alignment of the joint; and the determining the surgical plan is based on the biomechanical model.

In some embodiments, the surgical plan is determined to achieve one or more post-operative target joint parameters.

In some embodiments, the target joint parameters include a desired isometry profile of the joint; and the joint measurement data set includes an initial isometry profile of the joint.

In some embodiments, the target joint parameters include an alignment of the joint.

In some embodiments, the target joint parameters include a spacing of the first bone and the second bone at the joint.

In some embodiments, the target joint parameters include a desired loading or unloading of the joint.

In some embodiments, the placement of the first support on the first bone includes a position of one or more anchor points of the first support on the first bone; and the placement of the second support on the second bone includes a position of one or more anchor points of the second support on the second bone.

In some embodiments, the operating parameter of the linking member includes a characteristic affecting the energy absorption of the linking member.

In some embodiments, the operating parameter includes a stiffness or a tension of the linking member.

In some embodiments, the implant parameters further include a placement of: one or more fixation devices configured to couple the first support to the first bone; and one or more fixation devices configured to couple the second support to the second bone.

In some embodiments, the placement of the one or more fixation devices includes at least one of: a position of the one or more fixation devices; an orientation of the one or more fixation devices; or a depth of insertion of the one or more fixation devices into the first bone or the second bone.

In some embodiments, the localization system is configured to generate the joint measurement data set by localizing the one or more trackers coupled to at least one of the first bone or the second bone when the joint is in one or more constrained positions.

In some embodiments, the processing device is further configured to: based on the joint measurement data set, determine a biomechanical model of the joint, wherein the biomechanical model accounts for the effects of laxities and soft tissues on the alignment of the joint; and determine the surgical plan based on the biomechanical model.

In some embodiments, the control unit is further configured to select, for each fixation device hole, a fixation device reference from a plurality of fixation device references, each fixation device reference defining a diameter and a length of a respective fixation device, the selected fixation device reference having diameter is less than or equal to a diameter of the fixation device hole and a length corresponding to the respective predetermined depth of the fixation device In some embodiments, the control unit is configured to compute a representation of a relative pose of the surgical tool with respect to the predetermined drilling axis, a representation of the predetermined drilling axis, or a representation of the predetermined depth on a model of the first bone or the second bone; and a user interface is configured to display the representation.

In some embodiments, the user interface is further configured to display, for each fixation device hole, a representation of the selected fixation device reference.

In some embodiments, the control unit is configured to allow an activation of the surgical tool only when the surgical tool is aligned with the predetermined drilling axis.

In some embodiments, the control unit is configured to control the robotic arm to displace and to align the surgical tool at a relative speed with respect to the predetermined drilling axis between a predetermined minimum relative speed and a predetermined maximum relative speed.

In some embodiments, the control unit is configured to control the robotic arm to displace and to align the surgical tool at a rotation speed between a predetermined minimum rotation speed and a predetermined maximum rotation speed.

In some embodiments, the control unit is configured to control the robotic arm to displace and to align the passive tool guide in line with the configured drilling axis for each of the programmed holes.

In some embodiments, the robotic arm includes at least six motorized joints, and the control unit is configured to control a motorized joint of the at least six motorized joints

All patents and publications mentioned in this specification are herein incorporated by reference to the same extent as if each independent patent and publication was specifically and individually indicated to be incorporated by reference.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of any appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by any appended claims and the applicable rules of law.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one nonlimiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y herein should be interpreted as meaning either “X” or “Y” individually, or both “X and Y” together.

1. A method, comprising:
acquiring a joint measurement data set for a joint, wherein:
the joint is defined by a first bone and a second bone; and
the joint measurement data set comprises at least one of:
data on kinematics of the joint; or
data on laxities of the joint; and
based on the joint measurement data set, determining at least one target implant parameter of an implant configured to be coupled to the first bone and the second bone, wherein:
the implant comprises:
a first support configured to be fixed to the first bone;
a second support configured to be fixed to the second bone; and
a linking member extending between the first support and the second support; and
the implant is configured to, when implanted on the joint according to the at least one target implant parameter, achieve at least one of a desired alignment, a desired spacing, or a desired loading of the joint.

2. The method of claim 1, wherein the linking member is configured to absorb energy.

3. The method of claim 2, wherein:
the at least one target implant parameter comprises an operating parameter of the linking member; and
the operating parameter comprises a characteristic affecting energy absorption of the linking member.

4. The method of claim 1, wherein the at least one target implant parameter comprises at least one of a placement of the first support on the first bone or a placement of the second support on the second bone.

5. The method of claim 4, wherein:
the placement of the first support on the first bone comprises a pose of the first support on the first bone; and
the placement of the second support on the second bone comprises pose of the second support on the second bone.

6. The method of claim 4, wherein:
the placement of the first support on the first bone comprises a pose of one or more anchor points of the first support on the first bone; and
the placement of the second support on the second bone comprises a pose of one or more anchor points of the second support on the second bone.

7. The method of claim 6, wherein determining the pose of the one or more anchor points of the first support on the first bone or the pose of the one or more anchor points of the second support on the second bone comprises constraining the pose of the one or more anchor points of the first support or the one or more anchor points of the second support to avoid limiting zones defined by one or more soft tissues or bones surrounding the joint.

8. The method of claim 1, wherein the joint measurement data set comprises the data on kinematics of the joint, and wherein acquiring the data on kinematics of the joint comprises:
manipulating at least one of the first bone or the second bone in a natural range of motion; and
tracking a pose of the first bone or the second bone while manipulating the first bone or the second bone.

9. The method of claim 1, wherein the joint measurement data set comprises the data on kinematics of the joint, and wherein acquiring the data on kinematics of the joint comprises:
acquiring at least partial models of the first bone or the second bone; and
applying a biomechanical model to the at least partial models of the first bone or the second bone, wherein the biomechanical model is adapted to patient-specific geometry of the first bone or the second bone.

10. The method of claim 1, wherein the joint measurement data set comprises the data on laxities of the joint, wherein acquiring the data on laxities of the joint comprises:
manipulating at least one of the first bone or the second bone in a constrained range of motion; and
tracking a pose of the first bone or the second bone while manipulating the first bone or the second bone.

11. The method of claim 1, wherein the joint measurement data set comprises the data on laxities of the joint, wherein acquiring the data on laxities of the joint comprises:
acquiring at least partial models of the first bone or the second bone; and
applying a biomechanical model to the at least partial models of the first bone or the second bone, wherein the biomechanical model is adapted to patient-specific geometry of the first bone or the second bone.

12. The method of claim 1, wherein determining the at least one target implant parameter comprises:
determining, from the joint measurement data set, a pair of anisometric points (PAP) comprising a distance that satisfy a PAP length criteria, wherein the PAP comprises a first point on the first bone and a second point on the second bone, and wherein the distance of the PAP is a distance between the first point and the second point as the joint is manipulated in natural ranges of motion, wherein the at least one target implant parameter is determined based on the PAP comprising the distance that satisfies the PAP length criteria.

13. The method of claim 12, wherein the joint measurement data set comprises the data on laxities of the joint, and wherein the PAP length criteria is determined based on the data on laxities of the joint.

14. The method of claim 1, further comprising obtaining at least one of a partial model of the first bone or a partial model of the second bone, wherein determining the at least one target implant parameter is further based on the at least one of a partial model of the first bone or a partial model of the second bone.

15. The method of claim 1, further comprising obtaining a model of the implant, wherein determining the at least one target implant parameter is further based on the model of the implant.

16. The method of claim 1, further comprising obtaining the desired alignment, the desired spacing, or the desired loading of the joint, wherein determining the at least one target implant parameter is further based on the desired spacing, or the desired loading of the joint.

17. The method of claim 1, further comprising:
acquiring real-time tracking data, the real-time tracking data comprising:
a pose of the first bone or the second bone; and at least one of:
a pose of a surgical instrument; or
a pose of a passive tool guide; and
instructing, one or more of:
a robotic system to drill one or more holes in the first bone or the second bone in accordance with the at least one target implant parameter and based on the real-time tracking data;
the robotic system, based on instructions from a user presented with the real-time tracking data, to change a pose of the surgical instrument or the passive tool guide relative to the first bone or the second bone to drill one or more holes in accordance with the at least one target implant parameter;
the robotic system to move, based on the at least one target implant parameter and the real-time tracking data, the passive tool guide to place the passive tool guide in a pose to guide the surgical instrument to drill one or more holes in the first bone or the second bone in accordance with the at least one target implant parameter; or
a handheld robotic system to align, based on the at least one target implant parameter and the real-time tracking data, the surgical instrument with the first bone or the second bone for drilling one or more holes in the first bone or the second bone in accordance with the at least one target implant parameter.

18. The method of claim 1, further comprising:
acquiring real-time tracking data, the tracking data comprising a pose of the first bone or the second bone, a pose of a surgical instrument, and a pose of the implant device relative to each other;
presenting, on a user interface, a display comprising a model of the implant, a model of the surgical instrument, and a model of at least one of the first bone or the second bone, in relation to each other in accordance with the real-time tracking data and the at least one target implant parameter,
wherein a user is enabled to adjust a pose of the surgical instrument relative to the first bone or the second bone based on the user interface.

19. A system, comprising:
at least one processor; and
at least one storage medium having encoded thereon executable instructions that, when executed by the at least one processor, cause the at least one processor to carry out a method, the method comprising:
acquiring a joint measurement data set for a joint, wherein:
the joint is defined by a first bone and a second bone; and
the joint measurement data set comprises at least one of:
data on kinematics of the joint; or
data on laxities of the joint; and
based on the joint measurement data set, determining at least one target implant parameter of an implant configured to be coupled to the first bone and the second bone, wherein:
the implant comprises:
a first support configured to be fixed to the first bone;
a second support configured to be fixed to the second bone; and
a linking member extending between the first support and the second support; and
the implant is configured to, when implanted on the joint according to the at least one target implant parameter, achieve at least one of a desired alignment, a desired spacing, or a desired loading of the joint.

20-34. (canceled)

35. The system of claim 19, further comprising a robotic arm having a surgical instrument positioned thereon, wherein the method further comprises instructing the robotic arm to move the surgical instrument relative to the first bone or the second bone in accordance with the at least one target implant parameter.

36. The system of claim 19, further comprising a robotic arm having a surgical instrument positioned thereon, wherein the method further comprises instructing the robotic arm to move the surgical instrument relative to the first bone or the second bone to drill one or more holes in the first bone or the second bone in accordance with the at least one target implant parameter.

37. The system of claim 19, further comprising a robotic arm having a passive tool guide positioned thereon, wherein the method further comprises instructing the robotic arm to move the passive tool guide to place the passive tool guide in a pose to guide a surgical instrument to drill one or more holes in the first bone or the second bone in accordance with the at least one target implant parameter.

38. The system of claim 19, further comprising a handheld robotic surgical tool, wherein the method further comprises aligning, based on the at least one target implant parameter, the handheld surgical tool with the first bone or the second bone for drilling one or more holes in the first bone or the second bone in accordance with the at least one target implant parameter.

39. At least one computer-readable storage medium having encoded thereon executable instructions that, when executed by at least one control circuit, cause the at least one control circuit to carry out a method, the method comprising:
acquiring a joint measurement data set for a joint, wherein:
the joint is defined by a first bone and a second bone; and
the joint measurement data set comprises at least one of:
data on kinematics of the joint; or
data on laxities of the joint; and
based on the joint measurement data set, determining at least one target implant parameter of an implant configured to be coupled to the first bone and the second bone, wherein:
the implant comprises:
a first support configured to be fixed to the first bone;
a second support configured to be fixed to the second bone; and
a linking member extending between the first support and the second support; and
the implant is configured to, when implanted on the joint according to the at least one target implant parameter, achieve one or more of a desired alignment, a desired spacing, or a desired loading of the joint.

40-54. (canceled)
